(19) United States
(12) Patent Application Publication
(10) Pub. No.: US 2018/0110770 A1
(43) Pub. Date: Apr. 26, 2018

(54) FERROPTOSIS AND GLUTAMINOLYSIS INHIBITORS AND METHODS OF TREATMENT

(71) Applicants: MEMORIAL SLOAN-KETTERING CANCER CENTER, New York, NY (US); NEW YORK UNIVERSITY, New York, NY (US)

(72) Inventors: Minghui GAO, New York, NY (US); Ravichandran RAMASAMY, Ardsley, NY (US); Xuejun JIANG, Cresskill, NJ (US)

(21) Appl. No.: 15/821,015

(22) Filed: Nov. 22, 2017

Related U.S. Application Data
(63) Continuation of application No. PCT/US2016/034230, filed on May 26, 2016.
(60) Provisional application No. 62/166,413, filed on May 26, 2015.

(51) Int. Cl. A61K 31/473; A61P 25/00; A61P 9/00; A61P 13/12; A61P 1/16

(52) U.S. Cl. CPC A61K 31/473; A61P 25/00; A61P 1/16; A61P 13/12; A61P 9/00

(57) ABSTRACT

The present invention is directed to ferroptosis and glutaminolysis inhibitors and to methods of treatment or prevention of an organ injury caused by ischemia-reperfusion in a subject in need thereof, the method comprising administering to the subject a therapeutically effective amount of a ferroptosis inhibitor or a glutaminolysis inhibitor.

### CROSS-REFERENCE TO RELATED APPLICATIONS

This application is a continuation of PCT/US2016/034230 filed May 26, 2016 and published on Dec. 1, 2016 as WO 2016/191520, which claims the priority of U.S. Provisional Application No. 62/166,413 filed on May 26, 2015, the entire contents of each are hereby incorporated in their entirety into the present disclosure.

### STATEMENT ON FEDERALLY SPONSORED RESEARCH OR DEVELOPMENT

This invention was made with government support under NIH Grant Numbers R01CA166413, R01HL102022, P01HL60901, and P01AG026467 awarded by the National Institutes of Health. The government has certain rights in the invention.

### SEQUENCE LISTING

This application contains a Sequence Listing, created on May 26, 2015; the file, in ASCII format, is designated 3314072A_sequencelisting and is 1.46 KB in size. The file is hereby incorporated by reference in its entirety into this application.

### FIELD OF THE INVENTION

This invention relates to ferroptosis and glutaminolysis inhibitors and to methods of treatment using ferroptosis inhibitors or glutaminolysis inhibitors.

### BACKGROUND OF THE INVENTION

Ferroptosis has emerged as a new form of regulated necrosis that is implicated in various human diseases. However, the mechanisms of ferroptosis are not well defined. This study reports the discovery of novel molecular components of ferroptosis and its intimate interplay with cellular metabolism and redox machinery. Nutrient starvation often leads to sporadic apoptosis. Strikingly, we found that upon deprivation of amino acids, a more rapid and potent necrosis process can be induced in a serum-dependent manner, which was subsequently determined to be ferroptosis. Two serum factors, the iron-carrier protein transferrin and amino acid glutamine, were identified as the inducers of ferroptosis. We further found that the cell surface transferrin receptor and the glutamine-fueled intracellular metabolic pathway, glutaminolysis, played crucial roles in the death process. Inhibition of glutaminolysis, the newly identified essential component of ferroptosis, can reduce heart injury triggered by ischemia-reperfusion, suggesting a potential therapeutic approach for treating related diseases.

In multicellular organisms, programmed cell death, particularly apoptosis, is frequently activated in a highly orchestrated manner to fulfill specific physiological functions (Budihardjo et al., 1999; Danial and Korsmeyer, 2004; Fuchs and Steller, 2011; Green and Kroemer, 2004; Thompson, 1995). In mammals, there are two major apoptotic pathways, the mitochondria-mediated intrinsic pathway and the death receptor-mediated extrinsic pathway. In both pathways, caspases are the molecular executioners of cell death, and caspase activation is required for most of the morphological features associated with apoptosis. Defects in apoptosis contribute to the development of numerous human diseases.

However, apoptosis is not the only mechanism for programmed cell death. Recent studies have led to the identification of several other cell death processes that appear to be programmed but distinctive from apoptosis (Bergsbaken et al., 2009; Blum et al., 2012; Vanden Berghe et al., 2014; Yuan and Kroemer, 2010). The RIP3-dependent necrosis pathway is one of such processes (Moriwaki and Chan, 2013; Vandenabeele et al., 2010). RIP3-dependent necrosis can be triggered by tumor necrosis factor-α (TNFα) and is mediated by a signaling cascade involving protein kinases RIP1(Degterev et al., 2008) and RIP3(Cho et al., 2009; He et al., 2009; Kaiser et al., 2011; Newton et al., 2014; Oberst et al., 2011; Zhang et al., 2009), leading to activation of the downstream necrotic response. Up to date, the precise physiological function of RIP3-dependent necrosis has not been unambiguously established. However, mounting evidence suggests that it may benefit the organism under various infectious or inflammatory conditions (Cho et al., 2009; He et al., 2009; Murphy et al., 2013; Sun et al., 2012).

Recently, another form of regulated necrosis, known as ferroptosis, has been identified. It was shown that a synthetic compound, erastin, can induce a form of non-apoptotic cell death that requires iron (thus the name ferroptosis) (Dixon et al., 2012; Yagoda et al., 2007). Subsequent studies demonstrate that erastin inhibits cystine import and downstream glutathione synthesis, thus leading to deregulated cellular redox homeostasis and ultimately cell death (Dixon et al., 2012; Yang et al., 2014). Ferroptosis inhibition has been shown to be effective in treating diseases such as ischemia/reperfusion-induced organ damage in experimental models (Friedmann Angeli et al., 2014; Linkermann et al., 2014). Further, because cancer cells harboring oncogenic Ras appear to be more sensitive to ferroptosis induction, this novel form of cell death has also being explored for cancer treatment (Yagoda et al., 2007; Yang et al., 2014). Although ferroptosis is strongly implicated in human diseases, currently the precise molecular mechanisms and biological functions of ferroptosis remain to be poorly understood.

This study reports the discovery of novel players and mechanisms for ferroptosis regulation, as well as an intimate functional interplay between ferroptosis and cellular metabolism. We identified transferrin and L-glutamine as novel extracellular regulators of ferroptosis. We also demonstrated that both transferrin transport and the cellular metabolic process glutaminolysis are essential for ferroptosis triggered by deprivation of full amino acids or of cystine alone. Further, we present evidence to support that glutaminolysis is a potential therapeutic target for treating heart injury caused by ischemia-reperfusion, likely due to the essential role of glutaminolysis in ferroptosis.

### SUMMARY OF THE INVENTION

The present invention is directed to a method of treatment or prevention of an organ injury caused by ischemia-reperfusion in a subject in need thereof, the method comprising administering to the subject a therapeutically effective amount of a ferroptosis inhibitor or a glutaminolysis inhibitor.

In another embodiment, the present invention is directed to a method of treatment or prevention of an organ injury caused by ischemia-reperfusion in a subject in need thereof, the method comprising administering to the subject a therapeutically effective amount of a compound of formula (I):

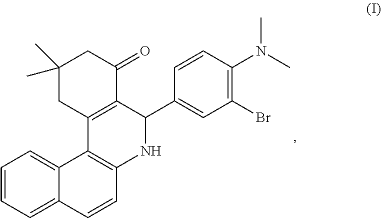 [SMILES: CN(C)c1ccc(C2Nc3ccc4ccccc4c3C3=C2C(=O)CC(C)(C)C3)cc1Br]

or a pharmaceutically acceptable salt thereof.

### BRIEF DESCRIPTION OF THE DRAWINGS

FIGS. 1A-1G show that serum induces potent non-apoptotic, RIP3-independent cell death upon amino acid starvation. FIG. 1A shows microscopy showing cell death induced by serum upon amino acid starvation. MEFs were treated as indicated for 12 hrs. Upper panel: phase-contrast, lower panel: propidium iodide (PI) staining. AA: amino acids, FBS: 10% (v/v) Fetal Bovine Serum. FIG. 1B shows quantitation of cell death by PI-staining coupled with flow cytometry. Cells were subjected to the same treatment as in FIG. 1A. Data are presented as mean±SD from representative experiment performed at least 3 times (***P<0.001 by unpaired Student t-test). FIG. 1C shows determination of cell viability by measuring cellular ATP levels. Cells were subjected to the same treatment as in FIG. 1A. FIG. 1D shows that serum-induced cell death is independent of caspase activation. MEFs were treated as indicated for 12 hrs and caspase-3 activation was assessed by immunoblotting. FIG. 1E shows that serum-induced cell death shows necrotic morphology. Representative still images from confocal time-lapse imaging of MEFs treated with amino acid starvation in the presence of FBS. The time after treatment is indicated. FIG. 1F shows that serum-induced cell death is independent of RIP3. RIP3+/+ MEFs and RIP3−/− MEFs were treated as indicated. Cell death was monitored by phase-contract microscopy and PI-staining. FIG. 1G shows that serum-induced cell death is independent of RIP3. RIP3+/+ MEFs and RIP3−/− MEFs were treated as indicated. Cell viability was determined by measuring cellular ATP levels.

FIGS. 2A-2C show that multiple serum components are required for starvation-induced cell death. FIG. 2A shows data for MEFs that were treated as indicated for 12 hrs. Upper panel: phase-contrast, Lower panel: PI staining. diFBS: 10% (v/v) dialyzed FBS, smFBS: 10% (v/v) Small molecule filtrates isolated from FBS. FIGS. 2B and 2C show data for MEFs that were treated as indicated for 12 hrs. Cell death was determined by PI staining coupled with flow cytometry (FIG. 2B), and cell viability was determined by measuring cellular ATP levels (FIG. 2C).

FIGS. 3A-3H show that transferrin and transferrin receptor are required for serum-dependent necrosis. FIG. 3A is a schematic showing the purification scheme for the death-inducing component in diFBS. FIG. 3B shows data where the final heparin column fractions were resolved by SDS-PAGE and stained with Coomassie blue. Arrow indicates the protein band correlating with killing activity. FIG. 3C shows that in amino acid-free medium, bax/bak-DKO MEFs were incubated with the heparin fractions in combination with smFBS as indicated, and cell death was determined by PI staining coupled with flow cytometry. FIG. 3D shows that immuno-depletion of transferrin (TF) abrogates the killing activity of serum. Serum was immuno-depleted with control IgG or anti-transferrin antibody (α-TF) as indicated, and subsequently used to induce cell death in Bax/bak-DKO MEFs under amino acid-free conditions for 12 hrs. Western blot was used to confirm the efficiency of transferrin depletion from FBS, with BSA as the loading control. FIG. 3E shows recombinant human holo-transferrin (rhTF) induced cell death in Bax/bak-DKO MEFs under amino acid-free conditions in a smFBS-dependent manner. FIG. 3F shows RNAi knockdown of transferrin receptor (TfR) inhibited serum-dependent necrosis. MEFs expressing control shRNA (NT) or two independent shRNAs targeting TfR were treated as indicated for 12 hrs and cell viability was measured. Western blot (lower panel) confirmed knockdown of TfR expression. FIG. 3G shows that iron-free bovine apo-transferrin (apo-bTF) did not have death-inducing activity. FIG. 3H shows that iron chelators blocked serum-dependent necrosis. MEFs were treated with 3 different iron chelators as indicated, and cell viability was determined by measuring cellular ATP levels. The following effective concentrations of the chelators were used: DFO (Deferoxamine) 80 μM, CPX (ciclopirox olamine): 10 μM, BIP (2,2-bipyridyl): 100 μM.

FIGS. 4A-4D show that glutamine is the death-inducing small molecule component in serum. FIG. 4A is a schematic showing the purification scheme for the death-inducing small molecule component in FBS. S: supernatant; P: pellet. See examples for detailed description. FIG. 4B shows LC/MS spectra of the active fraction from the XDB-C18 column. FIG. 4C shows that L-Gln (left panel) but not D-Gln (right panel) induces cell death in a diFBS-dependent manner under AA starvation. MEFs were treated as indicated for 12 hrs and cell viability was subsequently measured. FIG. 4D shows that L-Gln in combination with transferrin recapitulated the cell death-inducing activity of serum. bax/bak-DKO MEFs were treated as indicated for 12 hrs and cell viability was subsequently measured. L-Gln concentration, 0.1 mM. (*P<0.05,**P<0.01 ***P<0.001 by unpaired Student t-test)

FIGS. 5A-5E show that glutaminolysis mediates serum-dependent necrosis. FIG. 5A is a schematic overview of the glutaminolysis pathway. FIG. 5B shows that pharmacological inhibition of multiple components in the glutaminolysis pathway abrogated serum-dependent necrosis. The following inhibitors were used as indicated: L-Gln transporter inhibitor GPNA (5 mM); GLS inhibitor compound 968 (968, 20 μM); Pan-transaminases inhibitor AOA (0.5 mM). FIG. 5C shows that RNAi knockdown of SLC38A1 inhibited serum-dependent necrosis. Left: MEFs expressing non-targeting (NT) shRNA or shRNA targeting SLC38A1 were treated as indicated for 12 hrs and cell viability was subsequently measured. Right: qPCR measurement of SLC38A1 mRNA levels in MEFs infected with NT shRNA or shRNA targeting SLC38A1. FIG. 5D shows that Knockdown of GLS2 blocked serum-dependent necrosis. MEFs expressing non-targeting shRNA (NT) or two independent shRNAs targeting GLS2 were treated as indicated for 12 hrs and cell viability was subsequently measured. FBS: 5% (v/v). Western blotting (lower panel) confirmed the knockdown of GLS2 expression. FIG. 5E shows that GOT1 RNAi reduced serum-dependent necrosis. Left: MEFs expressing non-targeting (NT) shRNA or shRNA targeting GOT1 were treated as indicated for 12 hrs and cell viability was subsequently measured. Right: qPCR measurement of GOT1 mRNA levels in MEFs infected with NT shRNA or shRNA targeting GOT1. FIG. 5F shows that α-ketoglutarate can mimic the death-inducing activity of L-Gln but in a manner insensitive to the transaminase inhibitor AOA. MEFs were treated as indicated for 12 hrs, and cell viability was subsequently measured. α-KG: (Dimethyl-α-Ketoglutarate), 4 mM; AOA, 0.5 mM.

FIGS. 6A-6G show that cystine starvation and subsequent cellular redox homeostasis unbalance trigger serum-dependent necrosis. FIG. 6A shows that addition of cysteine (C, 0.2 mM) or cystine (CC, 0.2 mM) inhibited serum-dependent necrosis. MEFs were treated as indicated for 12 hrs. Cell viability was subsequently measured by flow cytometry. FIG. 6B shows that Cystine starvation alone is sufficient to induce cell death. MEFs were treated as indicated for 12 hrs and cell death was determined by PI staining coupled with flow cytometry. FIG. 6C shows that Glutathione (GSH) was depleted in the condition of serum-induced necrosis or cystine starvation. MEFs were treated as indicated for 4 hrs and harvested for total glutathione measurement. FIG. 6D shows accumulation of ROS in MEFs induced by serum under AA starvation condition (left) or cystine starvation (right). ROS levels in MEFs were determined by H₂DCFDA staining. H₂DCFDA assay was performed 8 hrs (Left) or 6 hours (right). FIG. 6E shows supplement of GSH blocked cell death induced by serum upon total AA starvation or cystine starvation. MEFs were treated as indicated for 12 hrs, and cell viability was subsequently measured by Flow Cytometry. FIG. 6F shows that cell death induced by serum upon AA starvation or cystine starvation can be prevented by antioxidant reagents NAC (0.2 mM) and Trolox (0.2 mM). MEFs were treated as indicated for 12 hrs, and cell viability was subsequently measured by flow cytometry. FIG. 6G shows blockage of GSH biogenesis by knocking down GCLC sensitized cell to cell death induced by serum upon AA starvation or cystine starvation. Left: MEFs expressing non-targeting (NT) shRNA or shRNA targeting GCLC were treated as indicated for 12 hrs and cell viability was subsequently measured by PI staining followed by flow cytometry. Right: qPCR measurement of GCLC mRNA levels in MEFs infected with NT shRNA or shRNA targeting GCLC.

FIGS. 7A-7H shows that serum-dependent necrosis is ferroptosis and is involved in ischemia-reperfusion heart injury. FIG. 7A shows that ferroptosis inhibitor Ferrastatin-1 (Fer-1) can inhibit serum-induced necrosis upon total AA starvation or cystine (CC) starvation. MEFs were treated as indicated for 12 hrs, and cell viability was subsequently measured by PI staining followed by flow cytometry. Erastin, 1 μM. FIG. 7B shows that erastin-induced ferroptosis required both transferrin and glutamine. bax/bak DKO MEFs were treated as indicated for 12 hrs, and cell viability was subsequently measured by PI staining followed by flow cytometry. Erastin: 1 μM. FIG. 7C shows that iron chelators inhibited erastin-induced ferroptosis. MEFs were treated as indicated for 12 hrs, and cell viability was subsequently measured by PI staining followed by flow cytometry. DFO, 80 μM; CPX, 10 μM Erastin 1 μM. FIG. 7D shows that inhibition of GLS by compound 968 (20 μM) or transaminases by pan-transaminases inhibitor AOA (0.5 mM) blocked erastin-induced ferroptosis. MEFs were treated as indicated for 12 hrs, and cell viability was subsequently measured by PI staining followed by flow cytometry. Erastin, 1 μM. FIG. 7E shows schematic protocol for the ischemia-reperfusion study involving 30-min of global ischemia followed by a 60-min reperfusion period. FIG. 7F shows determination of myocardial ischemic injury and function by left ventricular developed pressure (LVDP) recovery, showing improved functional recovery upon treatment with DFO or compound-968 (968). DFO: 80 μM. 968: 25 μM. *P<0.05, **P<0.01, ***P<0.001 by unpaired Student t-test, n=3-5 mice/group (same for FIGS. 7G and 7H). FIG. 7G shows representative cross-sections of hearts stained with TTC, demonstrating reduced infarct injury (pale region) upon DFO or 968 treatment. The plot (lower panel) shows quantification of infarct size of each group. FIG. 7H shows determination of myocardial ischemic injury by lactate dehydrogenase (LDH) release, demonstrating reduced infarct injury in the hearts treated with DFO or 968.

FIGS. 8A-8C show that serum induces potent non-apoptotic, RIP1-and RIP3-independent cell death upon amino acid starvation. FIG. 8A shows that caspase inhibitor zVAD cannot block serum-induced cell death. MEFs were treated with or without zVAD-FMK as indicated for 12 hrs. Cell viability was determined by measuring cellular ATP levels. Western blot showing that zVAD can block caspase-3 activation induced by UV light. FIG. 8B shows that Bax and Bak are not required for serum-induced cell death. bax/bak-DKO MEFs were treated as indicated for 12 hrs. Left: Cell death was quantitated by PI staining followed by flow cytometry. Right: Cell viability was determined by measuring cellular ATP levels. diFBS: dialyzed FBS, which did not induce cell death as full serum (FBS) did. FIG. 8C shows that RIP3 is required for TNFα-induced necrosis. RIP3+/+ or RIP3−/−MEFs were treated as indicated and cell viability was determined by measuring cellular ATP levels. Cycloheximide (Chx): 1 μg/ml; zVAD: 20 μM, TNFα: 100 ng/ml. Western blotting confirmed the knockout of RIP3.

FIGS. 9A-9H show that multiple types of cancerous and noncancerous cells can undergo serum-dependent necrosis. The results of 8 different cell lines (FIGS. 9A-9H) are resented. Cell death was measured by propidium iodide (PI) staining coupled with flow cytometry. diFBS: dialyzed FBS, which did not induce cell death as full serum (FBS) did.

FIG. 10 shows that iron-bound bovine holo-transferrin can induce cell death under amino acid starvation conditions in a smFBS-dependent manner. bak/bax-DKO MEFs were treated for 12 hrs as indicated. Cell viability was determined by measuring cellular ATP levels. holo-bTF: bovine holo-transferrin.

FIG. 11 shows that L-alanine-L-glutamine (A-Q) mimics the killing activity of L-glutamine. MEFs were treated as indicated for 12 hrs and cell viability was determined by measuring cellular ATP levels.

FIGS. 12A-12E show that glutaminolysis mediates serum-dependent necrosis. FIG. 12A shows that RNAi knockdown of SLC1A5 inhibited serum-dependent necrosis. Left: MEFs expressing non-targeting (NT) shRNA or shRNA targeting SLC1A5 were treated as indicated for 12 hrs and cell viability was subsequently measured. Right: qPCR measurement of SLC1A5 mRNA levels in MEFs infected with NT shRNA or shRNA targeting SLC1A5. FIG. 12B shows that GLS1 knockdown cannot block serum-dependent necrosis in MEFs. MEFs infected with NT shRNA or two independent shRNAs against GLS1 were treated as indicated for 12 hrs and cell viability was subsequently measured. Western blot (lower panel) confirmed the knockdown of GLS1 expression. FIG. 12C shows that GLS1 inhibitor BPTES failed to block serum-dependent necrosis in MEFs. MEFs were treated as indicated for 12 hrs, in the presence or absence of BPTES (Bis-2-(5-phenylacetamido-1,3,4-thiadiazol-2-yl)ethyl sulfide; 10 μM). FIG. 12D shows that GLUD1 is dispensable for serum-dependent necrosis in MEFs. MEFs infected with Non-Targeting (NT) shRNA or two independent shRNAs against GLUD1 were treated as indicated for 12 hrs, and cell viability was subsequently measured. FIG. 12E shows that AOA cannot inhibit TNF-α induced apoptosis. TS: TNF-α (50 ng/ml)+SMAC mimetic (0.5 μM); AOA: 0.5 mM. Data are presented as mean±SEM, n=3 (**P<0.01, ***P<0.001 by unpaired Student t-test).

FIGS. 13A-13D show that cystine but no other amino acid can inhibit serum-induced necrosis. MEFs were treated for 12 hrs as indicated. Cell viability was determined by PI staining coupled with flow cytometry.

FIGS. 14A-14F show that MEK inhibition is not sufficient to block ferroptosis, and the ferroptotic cell death-inducing activity of FBS from different sources correlates with seral L-glutamine concentrations. FIG. 14A shows that MEFs were treated as indicated for 12 hrs and cell viability was determined by measuring cellular ATP levels. MEFs were treated for 12 hrs as indicated. Cell viability was determined by PI staining coupled with flow cytometry. PD0325901, 10 μM, U0126, 10 μM. Western blotting confirmed the inhibition of MEK1/2 kinase activity by PD0325901 and U0126. FIG. 14B shows the antioxidant activity of different compounds. The in vitro antioxidant activity was analyzed by a 2,2-Diphenyl-1-picrylhydrazyl (DPPH) assay measuring reduction of DPPH. FIG. 14C shows that three independent sources of FBS (C, PAA, and CT) from different companies are competent to induce cell death of MEFs under amino acid-starvation conditions. MEFs were treated for 12 hrs as indicated. Cell viability was determined by measuring ATP level. FIG. 14D shows that two sources of FBS (s and g) are incompetent to induce cell death of MEFs under amino acid-starvation conditions. As diFBS, the killing activity of incompetent FBS can be restored by adding smFBS generated from competent FBS. MEFs were treated for 12 hrs as indicated. Cell viability was determined by measuring ATP level. FIG. 14E shows the concentration of L-Glutamine measured in both killing and non-killing FBS. FIG. 14F shows that the killing activity of incompetent FBS can be restored by supplementation of L-Gln (4 mM). MEFs were treated for 12 hrs as indicated. Cell viability was determined by measuring ATP level.

### DETAILED DESCRIPTION OF THE INVENTION

All patents, publications, applications and other references cited herein are hereby incorporated in their entirety into the present application.

In practicing the present invention, many conventional techniques in molecular biology, microbiology, cell biology, biochemistry, and immunology are used, which are within the skill of the art. These techniques are described in greater detail in, for example, Molecular Cloning: a Laboratory Manual 3rd edition, J. F. Sambrook and D. W. Russell, ed. Cold Spring Harbor Laboratory Press 2001; Recombinant Antibodies for Immunotherapy, Melvyn Little, ed. Cambridge University Press 2009; “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001). The contents of these references and other references containing standard protocols, widely known to and relied upon by those of skill in the art, including manufacturers' instructions are hereby incorporated by reference as part of the present disclosure.

In the description that follows, certain conventions will be followed as regards the usage of terminology. Generally, terms used herein are intended to be interpreted consistently with the meaning of those terms as they are known to those of skill in the art.

Nutrient availability is one of the key parameters for cells to make life-or-death decisions. It has been documented that long-term deprivation of growth factors, amino acids, or glucose causes gradual cell death (Wei et al., 2001). Although apoptotic machinery is often elicited in such starvation-induced death, this nevertheless can be considered a passive death process due to failure of the cell to survive the stressful conditions of nutrient/growth factor deprivation.

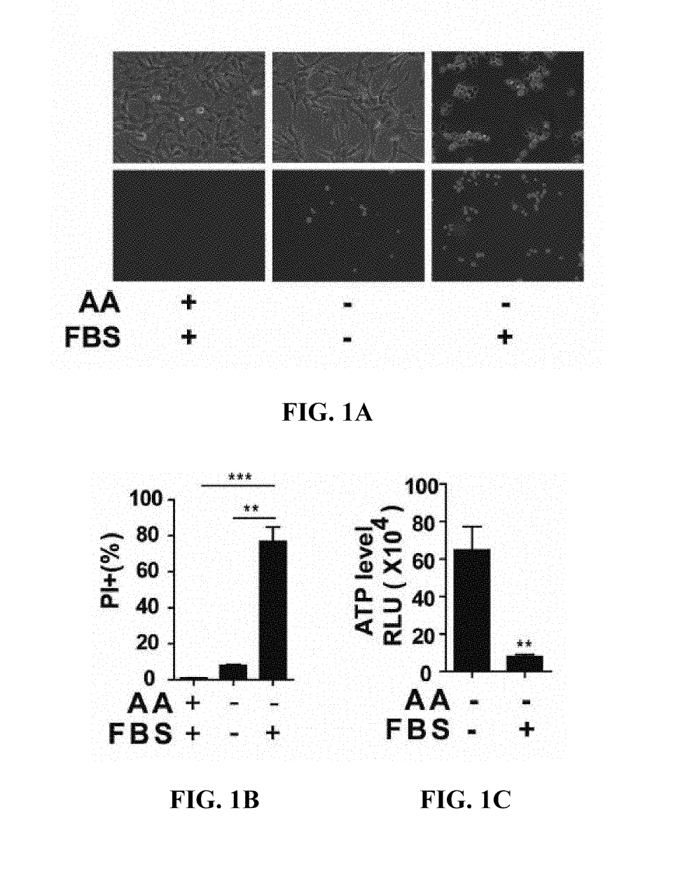

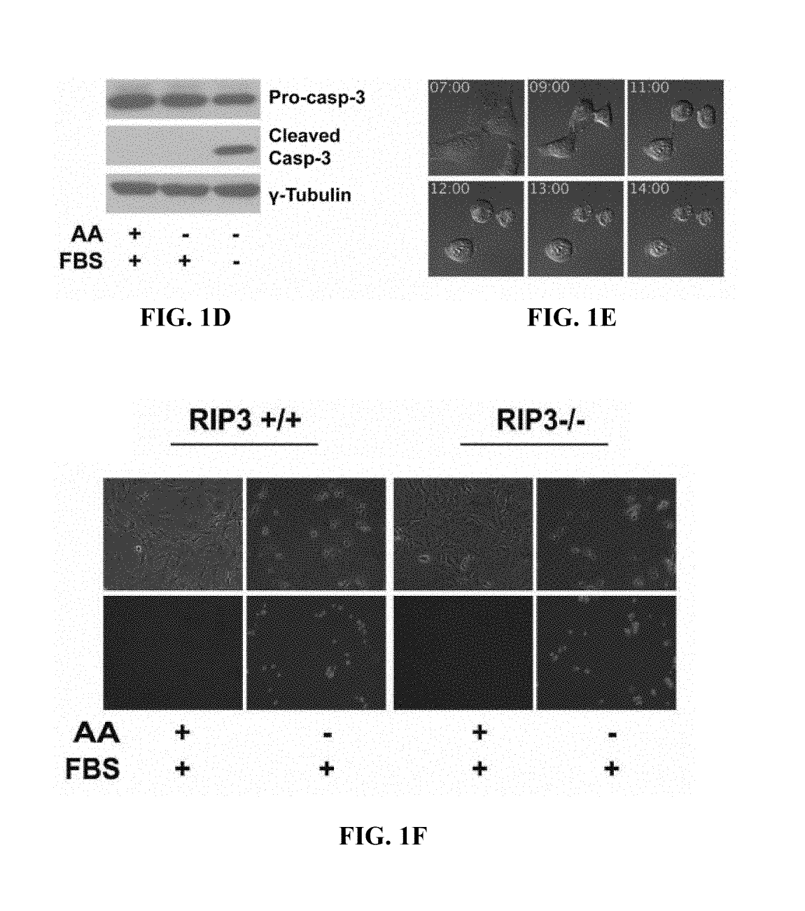

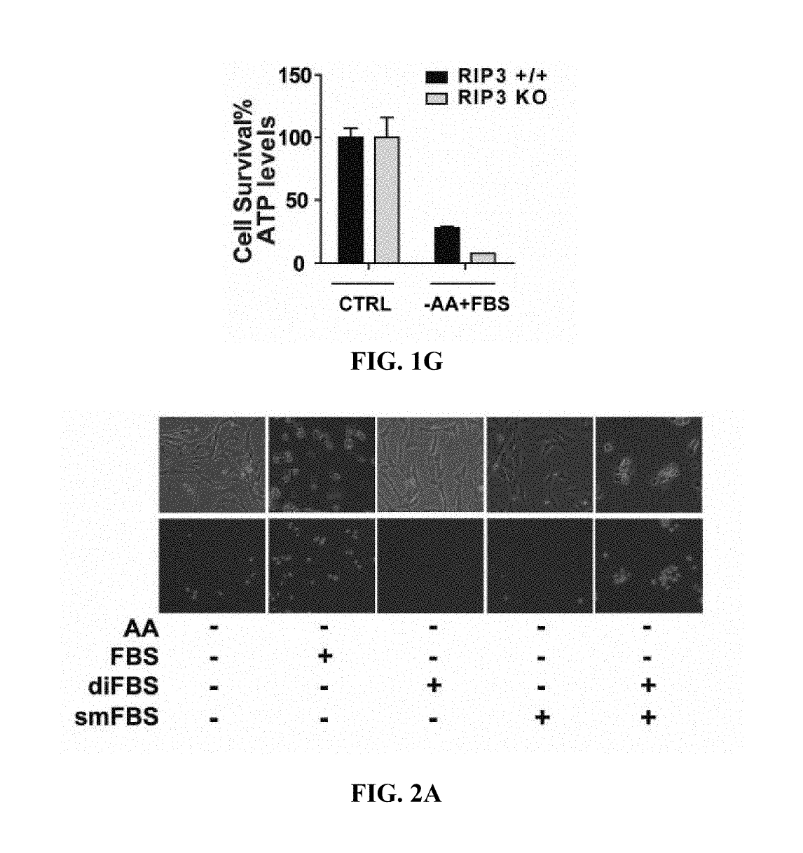

To recapitulate cell death under nutrient/growth factor deprivation, we incubated mouse embryonic fibroblasts (MEFs) in growth medium containing glucose but lacking amino acids and serum. Modest cell death was observed after 12 hrs (FIG. 1A). We then incubated MEFs in amino acid-free medium containing full serum, expecting that growth factors in serum would mitigate cell death. Surprisingly, we observed much more potent cell death (FIG. 1A), which was further confirmed by propidium iodide (PI) staining (FIGS. 1A, 1B) and measurement of cellular ATP levels (FIG. 1C).

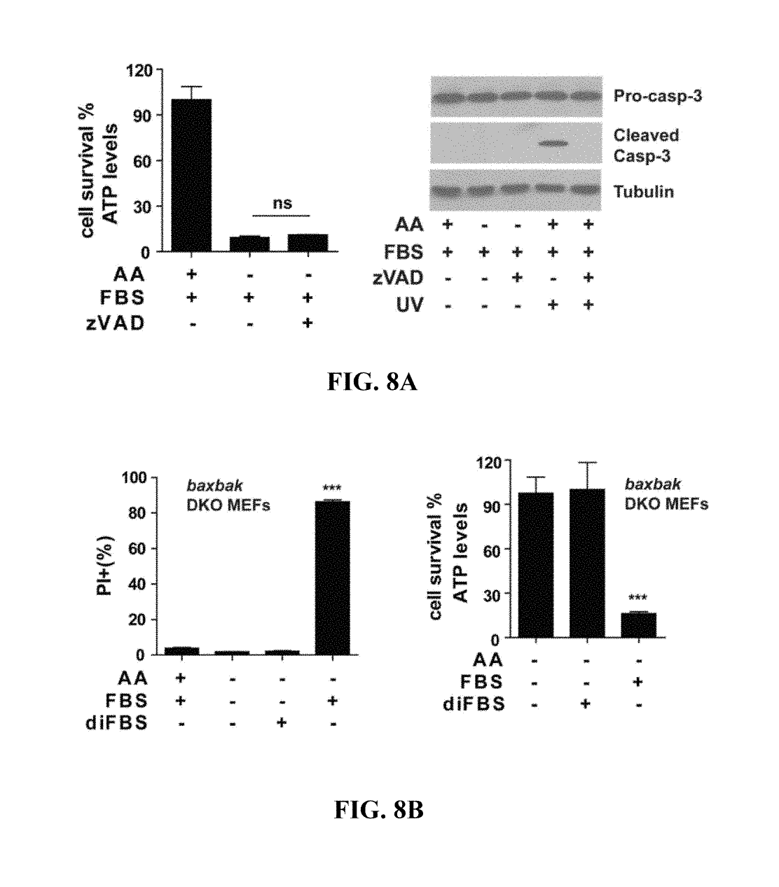

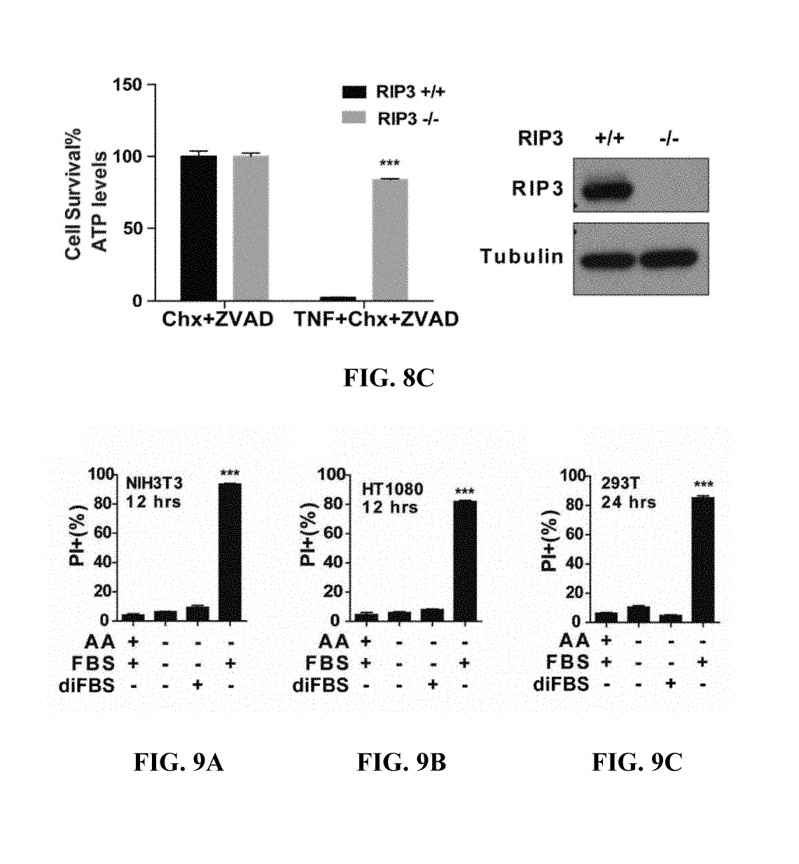

We subsequently investigated the molecular nature of this serum-induced cell death process. Cell death induced by combined deprivation of amino acids and serum was typical apoptosis, associated with caspase activation (FIG. 1D) and characteristic morphological changes, such as chromosomal condensation, membrane blebbing, and formation of apoptotic bodies. However, in the presence of serum, although cell death was significantly more potent, there was no caspase activation (FIG. 1D). Consistently, use of the pan-caspase inhibitor zVAD-FMK or deletion of bax and bak, genes essential for mitochondria-mediated apoptosis, failed to block such serum-induced cell death (FIGS. 8A, 8B). Further, the morphological changes associated with this cell death process were distinct from apoptosis (FIG. 1E). These results demonstrate that upon amino acid starvation, serum can induce non-apoptotic cell death in MEFs.

Through live cell time-lapse imaging, we observed that this cell death process shared key morphological features with necrosis, including cell rounding, swelling and plasma membrane rupture (FIG. 1E). Recent studies have established a programmed necrosis pathway dependent on the protein kinase RIP3 (Cho et al., 2009; He et al., 2009; Zhang et al., 2009). However, RIP3 gene deletion did not prevent serum-induced cell death in MEFs, although it completely blocked TNFα-induced necrosis as predicted (FIGS. 1F, 1G and FIG. 8C). These results indicate that the RIP3 -dependent necrosis pathway is not responsible for this novel serum-induced cell death.

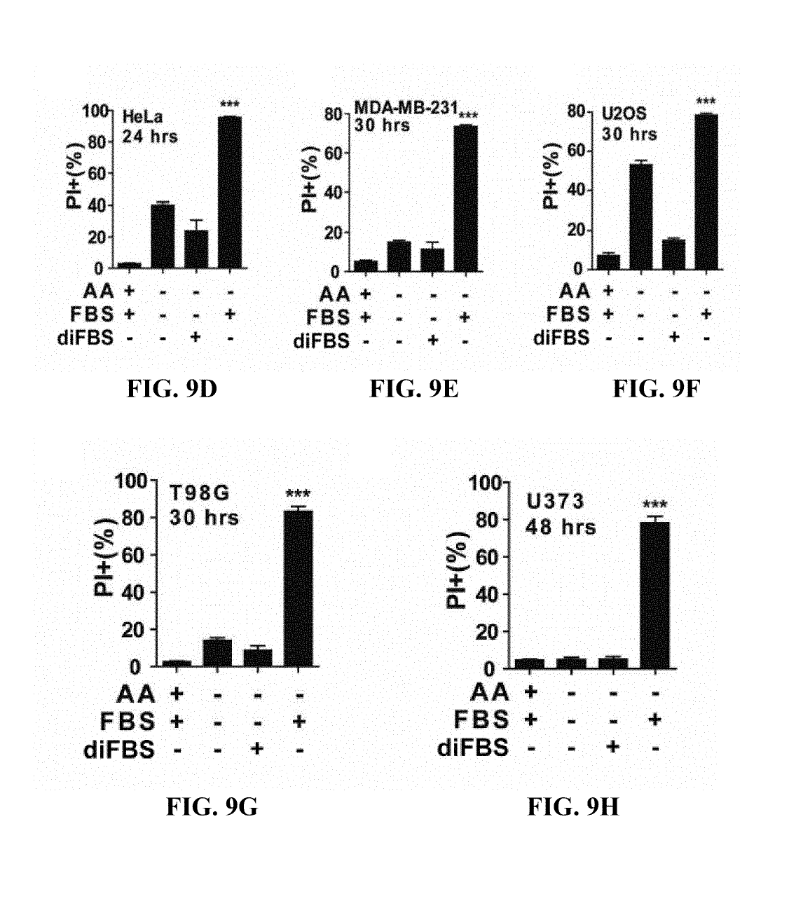

Importantly, induction of potent cell death by serum under the condition of amino acid starvation can be observed in various types of cancerous and noncancerous cells. While amino acid/serum-double starvation could induce different levels of death in a time-and cell type-dependent manner, addition of serum unanimously further potentiated cell death in these cells (FIG. 9).

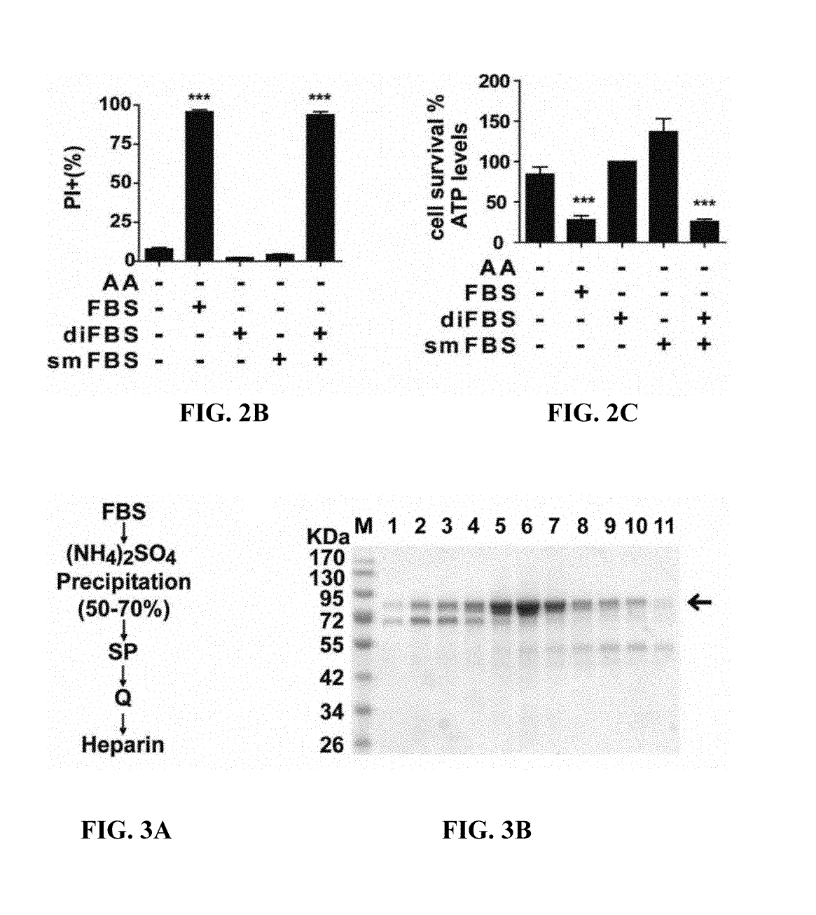

The results (FIG. 1) indicated that certain serum factor(s) is responsible for the activation of this type of cell death in a RIP3-independent, non-apoptotic manner. Biologically active components in serum include both macromolecules (such as proteins) and small molecules. We removed small molecules in fetal bovine serum (FBS) by dialysis and then tested whether the dialyzed FBS (diFBS) can induce cell death upon amino acid starvation. Unlike full FBS, diFBS failed to induce potent cell death, and it even protected against the modest apoptotic cell death induced by amino acid/serum-double starvation in MEFs (FIG. 2), presumably due to pro-survival growth factors presented in serum. The small molecule fraction of FBS (smFBS) prepared by filtering FBS through a size-exclusion membrane also failed to mimic the death-inducing activity of FBS. Only a combination of diFBS with smFBS fully restored the potent killing (FIG. 2). Therefore, multiple serum factors, of both macromolecule and small molecule natures, are required to induce this form of necrosis upon amino acid starvation.

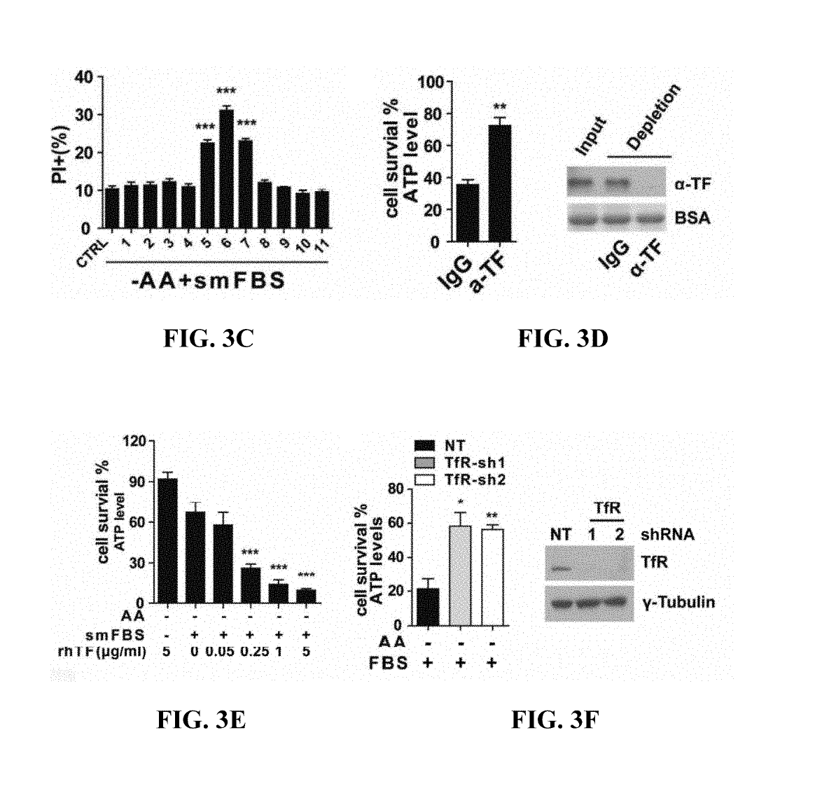

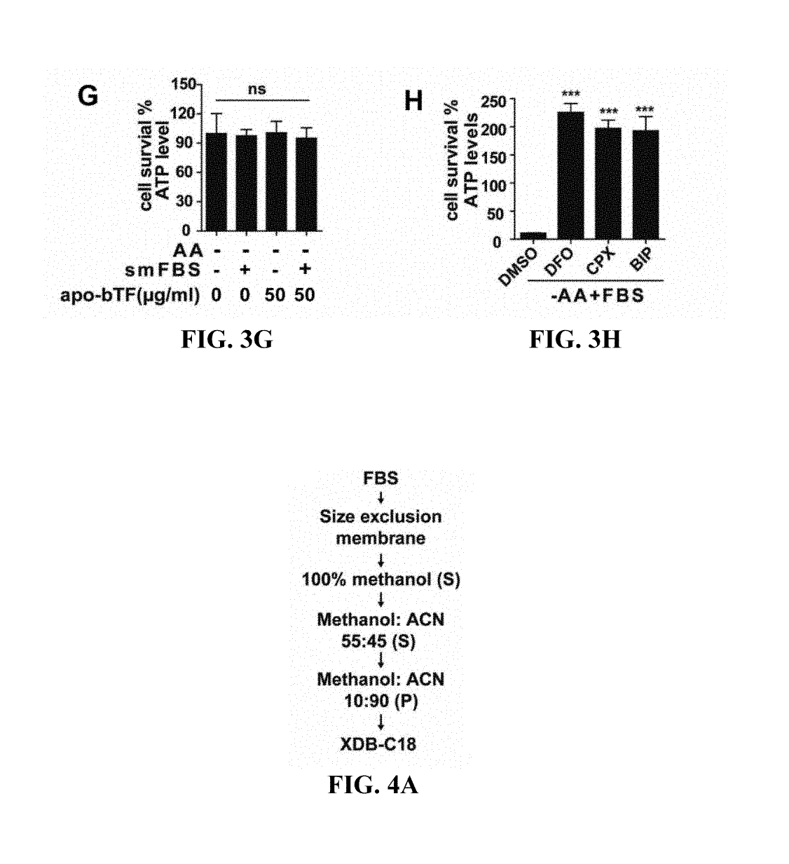

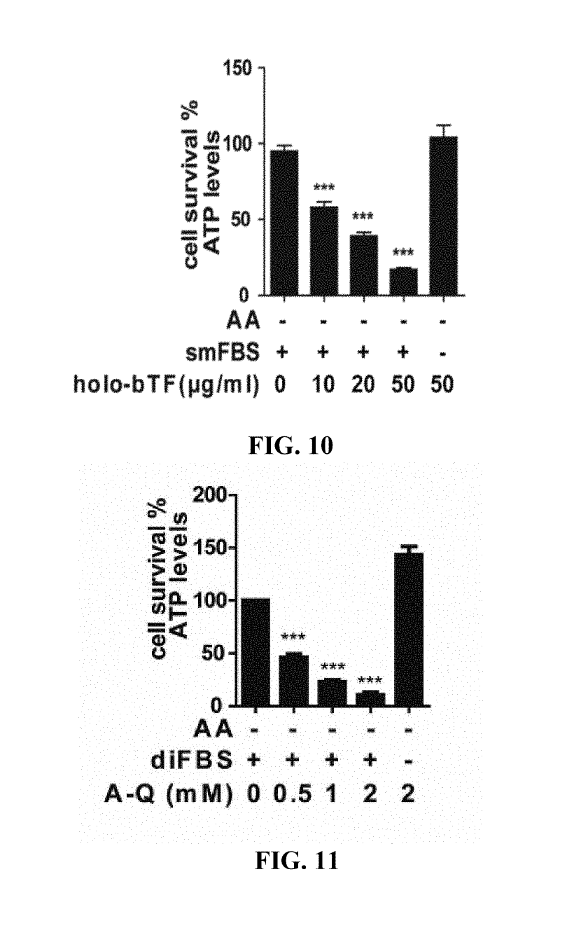

To identify the active component(s) in diFBS, we fractionated diFBS by ammonium sulfate precipitation in combination with various chromatographic columns. The killing activity of each fraction was monitored by incubating the fraction with cultured cells freshly switched to amino acid/serum-free medium, supplemented with the smFBS fraction. Cell death was measured after incubation for 12 hrs. In this assay, we used bax/bak-double knockout (DKO) MEFs to avoid amino acid starvation-induced apoptosis. After a four-step fractionation procedure (FIG. 3A), we purified a single protein (FIG. 3B) that correlated with killing activity (FIG. 3C). Mass spectrometry analysis revealed the identity of the protein to be bovine transferrin. To validate this novel activity of transferrin, we immuno-depleted transferrin from FBS, and found that the death-inducing activity of FBS was indeed dramatically decreased (FIG. 3D). Further, upon amino acid starvation, addition of commercial bovine holo-transferrin induced robust cell death in the presence of smFBS (FIG. 10). The effective concentration of transferrin in these assays was well within the range of serum transferrin concentration (0.49-2.63 mg/ml) (Valaitis and Theil, 1984). To rule out the possibility that the killing activity came from certain serum molecules co-purified with transferrin instead of transferrin per se, we tested recombinant human holo-transferrin that was expressed in rice (thus serum was not involved in the expression and purification). Again, upon amino acid starvation and in the presence of smFBS, recombinant human holo-transferrin could induce cell death in a dose-dependent manner (FIG. 3E).

Transferrin is an iron carrier protein in serum that can be transported into the cell via receptor-mediated endocytosis (Andrews and Schmidt, 2007). To determine whether transferrin needs to be imported into the cell to exert its death-inducing function, we first tested the requirement of transferrin receptor (TfR) for serum-induced cell death. Indeed, when TfR expression was knocked down by RNAi, cell death was significantly inhibited (FIG. 3F). Transferrin can only interact with TfR and be transported into the cell when it is loaded with iron. Consistently, iron-free bovine apo-transferrin does not possess killing activity (FIG. 3G). Further, we found that multiple iron chelators can inhibit cell death (FIG. 3H), which is in agreement with the requirement of transferrin import for this mode of cell death.

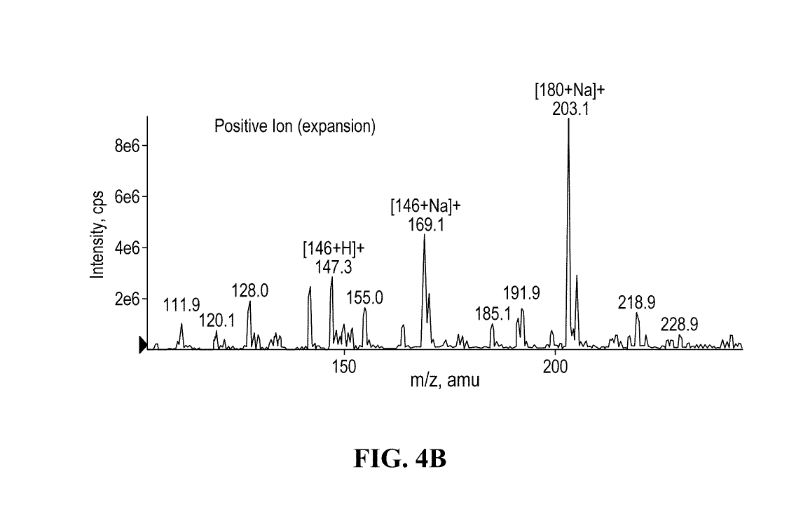

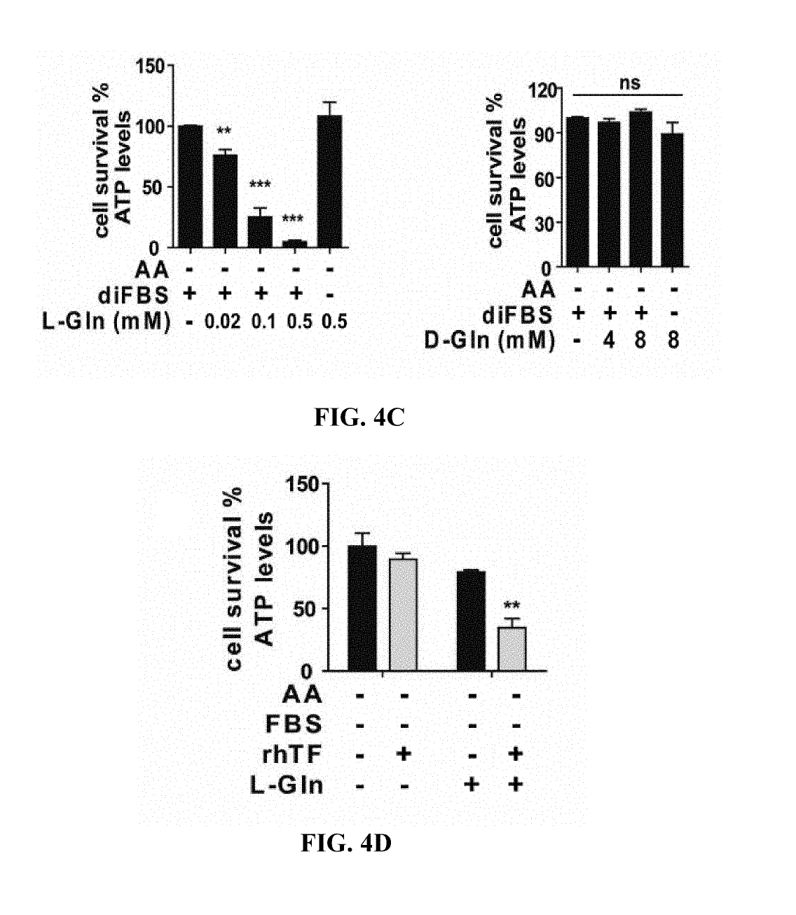

We next sought to identify the small molecule component of FBS required for death induction. The smFBS fraction was subjected to multiple steps of fractionation (FIG. 4A). The resulting fractions of each step were assessed for the death-inducing activity in MEFs in combination with diFBS. The active fractions obtained from the last step of purification (reverse phase-C18 HPLC) were analyzed by mass spectrometry, revealing several mass peaks in the fractions possessing the killing activity (FIG. 4B). Three major mass peaks showed mass equal to that of two abundant components of serum: L-glutamine (146 Da, with Na⁺ or H⁺) and glucose (180 Da, with Na⁺) (FIG. 4B). Glucose failed to recapitulate the killing activity in combination with diFBS or transferrin, which was expected, because the cells were cultured in high-glucose medium, and diFBS alone could not induce cell death upon amino acid starvation.

To determine whether glutamine (Gin) plays a role in serum-dependent necrosis, we compared L-Gln with several related amino acids. In a dose-dependent manner and upon amino acid starvation, L-Gln but not D-Gln or other tested amino acids induced potent cell death in the presence of diFBS (FIG. 4C). Because L-Gln tends to degrade in medium, to rule out the possibility that the killing activity is due to a degraded product rather than L-Gln itself, we tested a chemically stable L-Gln replacement, L-alanine-L-glutamine (A-Q). A-Q in combination with diFBS was also able to induce cell death upon amino acid starvation (FIG. 11). Further, as expected, the requirement of diFBS in this assay can be replaced with transferrin (FIG. 4D). In this later experiment, bax/bak-DKO MEFs were used to avoid apoptosis induced by amino acid/serum-double starvation. It should be noted that unlike in wild-type (WT) MEFs, L-Gln alone induced modest but measurable cell death in bax/bak-DKO MEFs, and addition of transferrin further enhanced cell death (FIG. 4D).

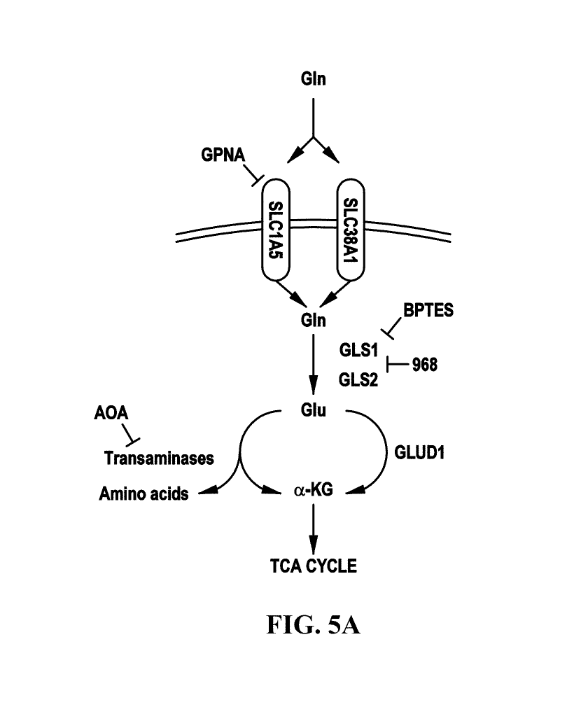

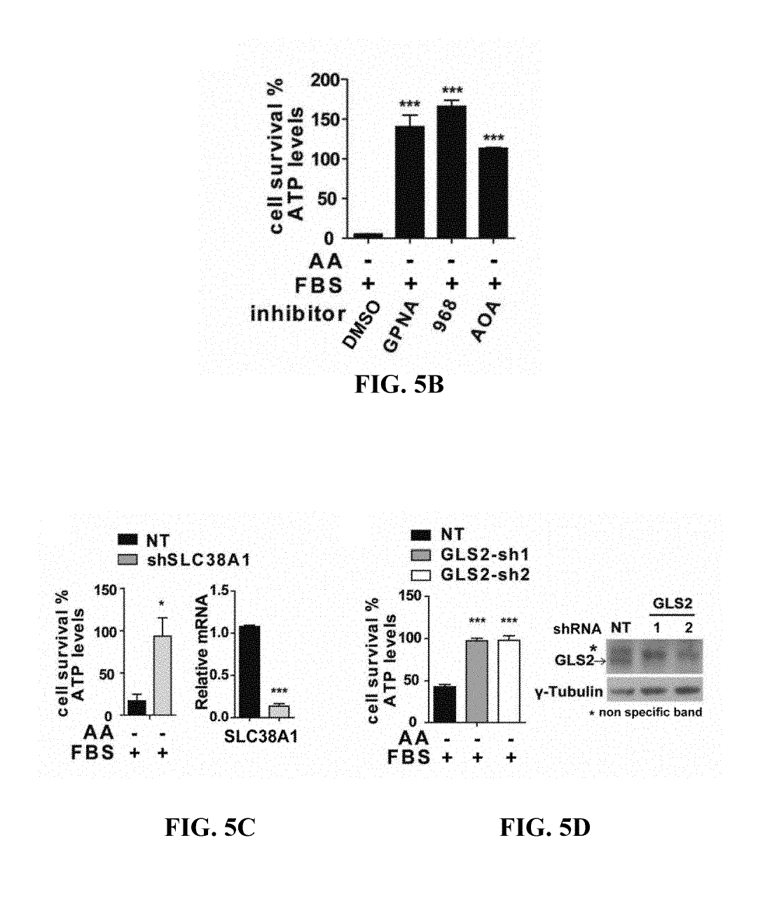

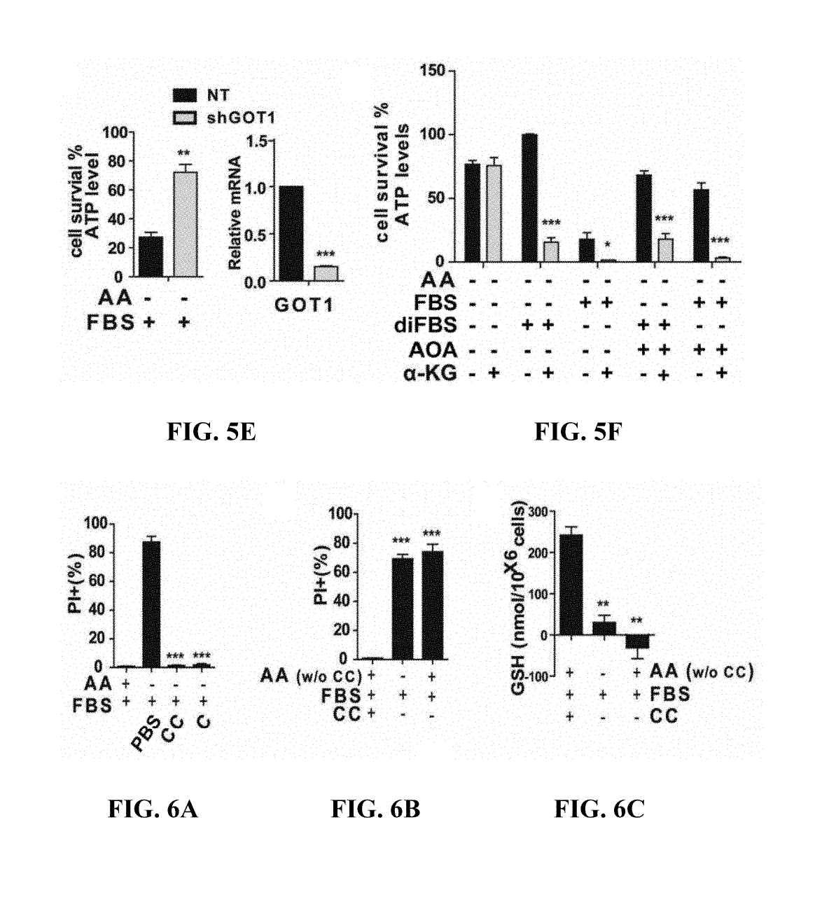

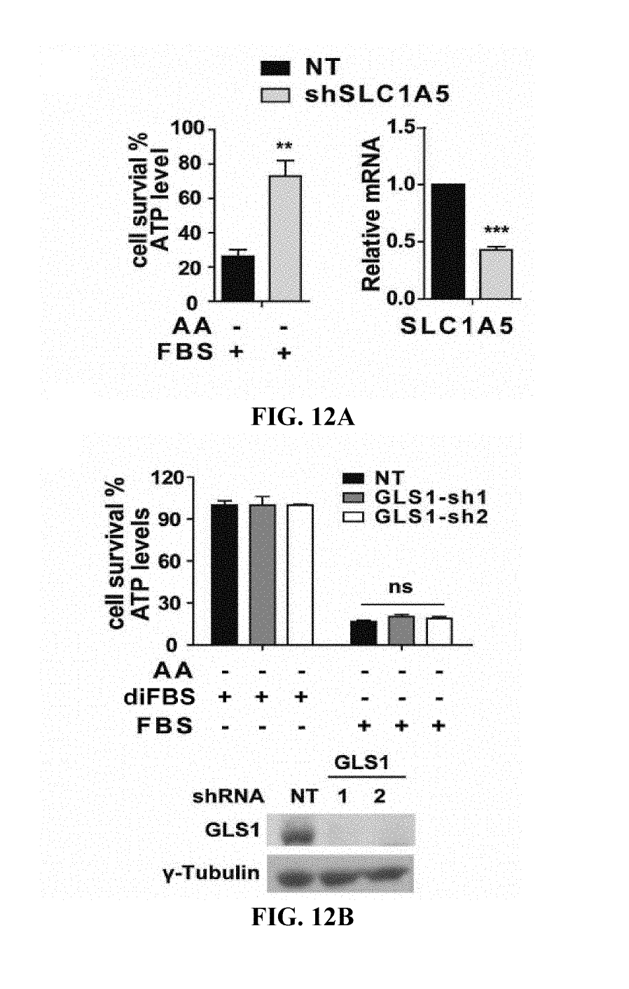

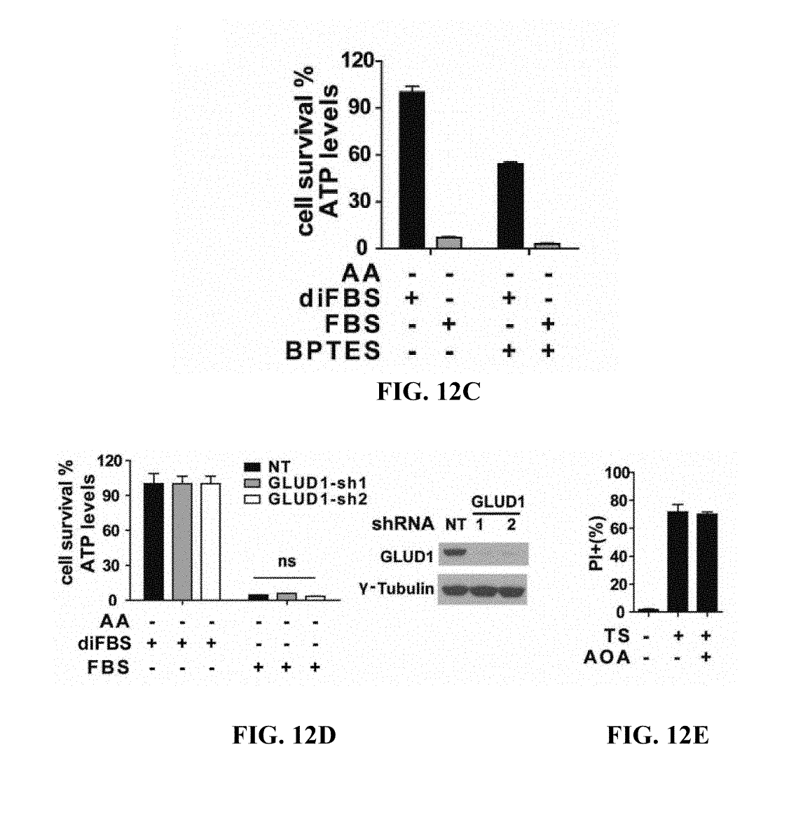

L-Gln is the most abundant amino acid in the body. Through glutaminolysis, proliferating cells use L-Gln both as a nitrogen source for the biosynthesis of nucleotides, amino acids, and hexamine, and as an important carbon source for the tricarboxylic acid (TCA) cycle (DeBerardinis et al., 2008). We sought to identify the molecular basis underlying the role of L-Gln and glutaminolysis in serum-dependent necrosis. L-Gln uptake is mainly dependent on receptors SLC1A5 and SLC38A1 (McGivan and Bungard, 2007) (FIG. 5A). We found that pharmacological inhibition of SLC1A5 by L-g-glutamyl-p-nitroanilide (GPNA) (Esslinger et al., 2005) or RNAi knockdown of SLC38A1 markedly blocked serum-dependent necrosis (FIGS. 5B, C). In cells, Gln is converted into glutamate (Glu) by glutaminases (GLS) (Curthoys and Watford, 1995). Compound 968 (968), an inhibitor of GLS (Wang et al., 2010), completely inhibited serum-dependent necrosis (FIG. 5B). There are two isoforms of mammalian GLS, GLS1 and GLS2 (Curthoys and Watford, 1995). We individually knocked down GLS1 and GLS2, and found that knockdown of GLS2 but not GLS1 significantly inhibited serum-dependent necrosis in MEFs (FIGS. 5D and 12A). Consistent with this result, Bis-2-(5 -phenylacetamido-1,3 ,4-thiadiazol-2-yl)ethyl sulfide (BETPS), a GLS1-specific inhibitor (Robinson et al., 2007), failed to block serum-dependent necrosis in MEFs (FIG. 12B).

Downstream of glutaminolysis, glutamate can be further converted into α-ketoglutarate (α-KG) either by glutamate dehydrogenase (GLUD1)-mediated glutamate deamination or by transaminases-mediated transamination (Hensley et al., 2013) (FIG. 5A). We found that transaminases but not GLUD1 are required for metaptosis in MEFs, as amino-oxyacetate (AOA), a pan inhibitor of transaminases (Wise et al., 2008), but not GLUD1 RNAi, could inhibit serum-dependent necrosis (FIG. 5B and FIG. 12C).

We then explored whether downstream metabolites of glutaminolysis can mimic the killing activity of L-Gln. Indeed, upon amino acid starvation, α-KG in combination with diFBS can induce potent cell death even in the presence of transaminase inhibitor AOA (FIG. 5E). This result is consistent with the fact that in the glutaminolysis pathway, α-KG is a downstream metabolite of transaminases, the target of AOA.

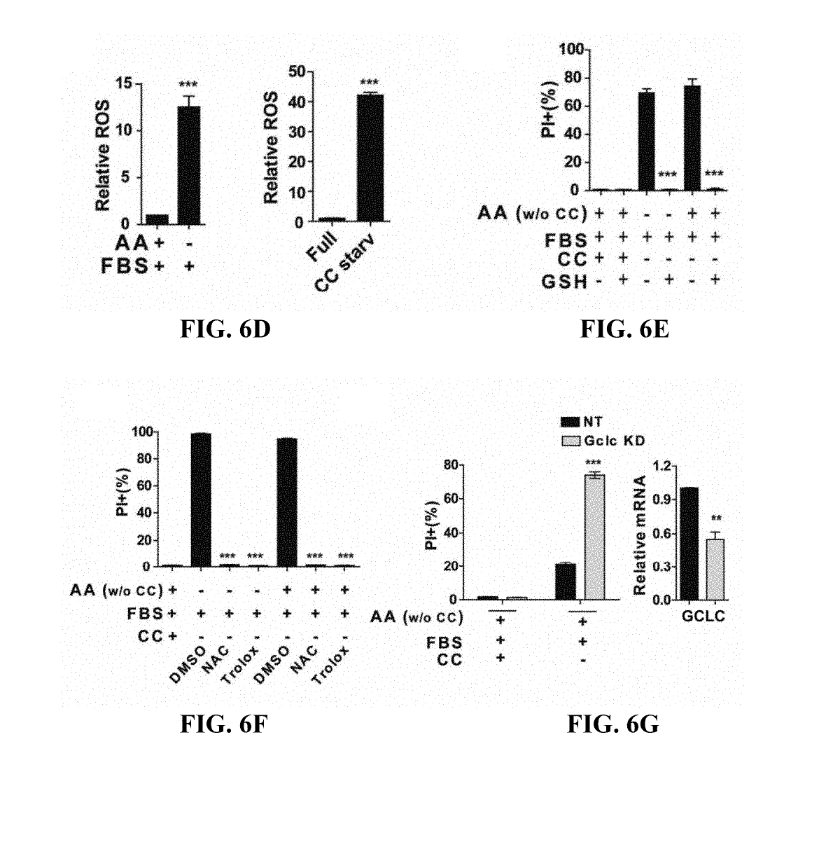

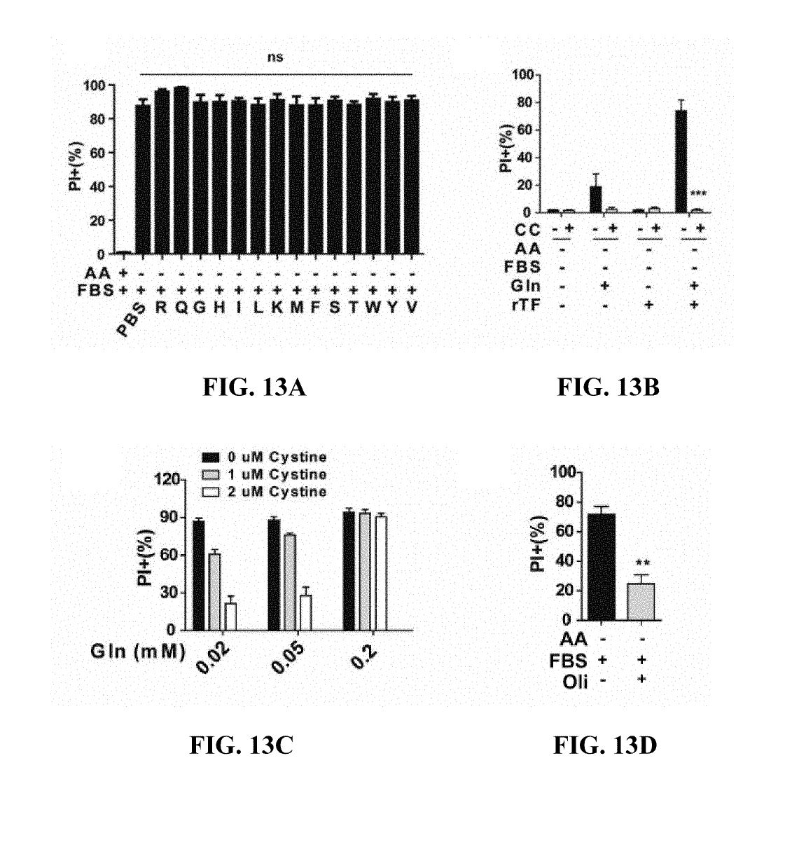

Does serum-dependent necrosis require deprivation of all amino acids, a specific group of amino acids, or a single amino acid? To address this question, we examined which amino acid(s) can rescue cells from full amino acid starvation-induced, serum-dependent necrosis. Adding back a single amino acid, cysteine or cystine, but no other amino acids, rescued cells from necrosis (FIG. 6A, FIGS. 13A-13D). Conversely, cystine starvation alone, in the presence of all other amino acids and serum, is sufficient to induce cell death (FIG. 6B). As a building block of the cellular reducing agent glutathione, cysteine is required for maintaining cellular redox homeostasis. Therefore, it is likely that cysteine starvation induces cell death via depleting cellular glutathione (GSH) and consequently increasing reactive oxygen species (ROS). For this reason, we determined the cellular GSH and ROS levels under conditions that trigger serum-dependent necrosis. Indeed, these conditions caused dramatic decrease of GSH level and increase of ROS level in cells (FIGS. 6C, 6D). Supplement of GSH or the biosynthetic precursor of GSH, N-acetylcysteine (NAC), or addition of ROS scavenger Trolox (6-hydroxy-2,5,7,8-tetramethylchroman-2-carboxylic acid) all effectively blocked cell death under these conditions (FIGS. 6E, 6F). To further confirm the critical role of glutathione synthesis in cell survival, we knocked down glutamate cysteine ligase catalytic subunit (GCLC), an essential enzyme for GSH synthesis. As expected, GCLC RNAi sensitized the cells to death induced by cystine starvation (FIG. 6G).

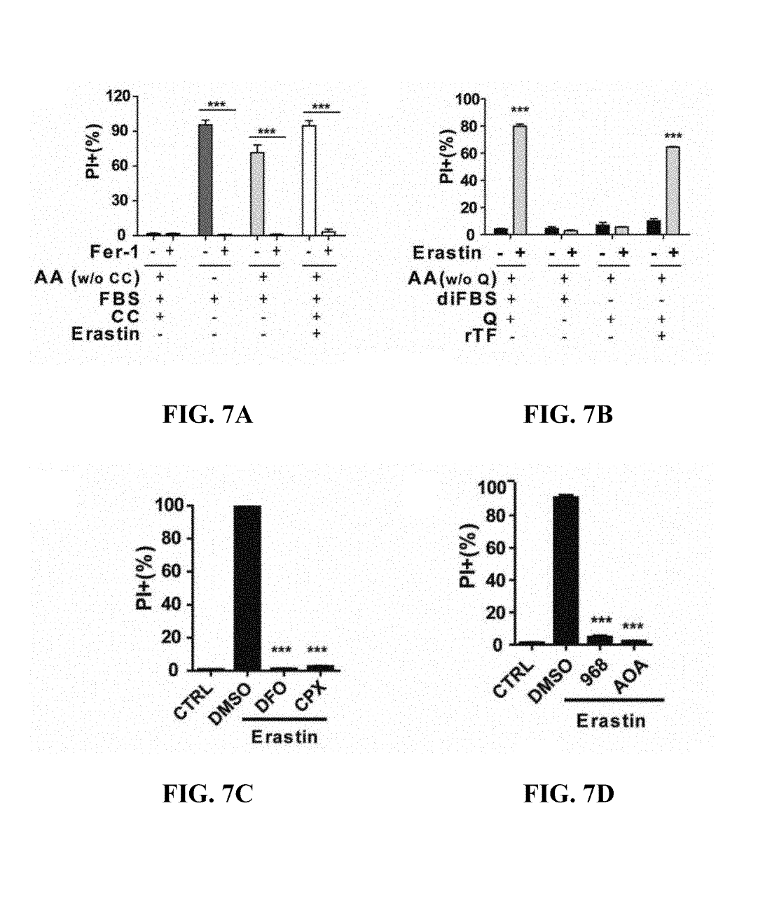

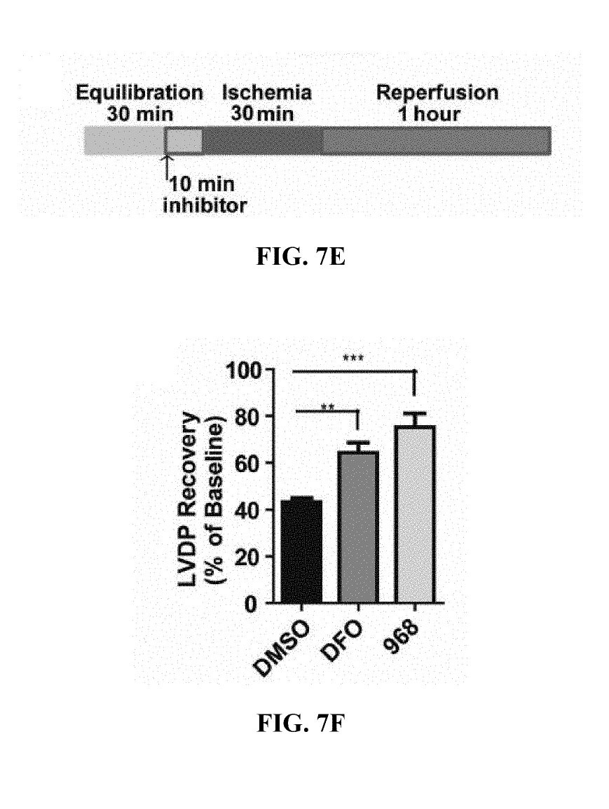

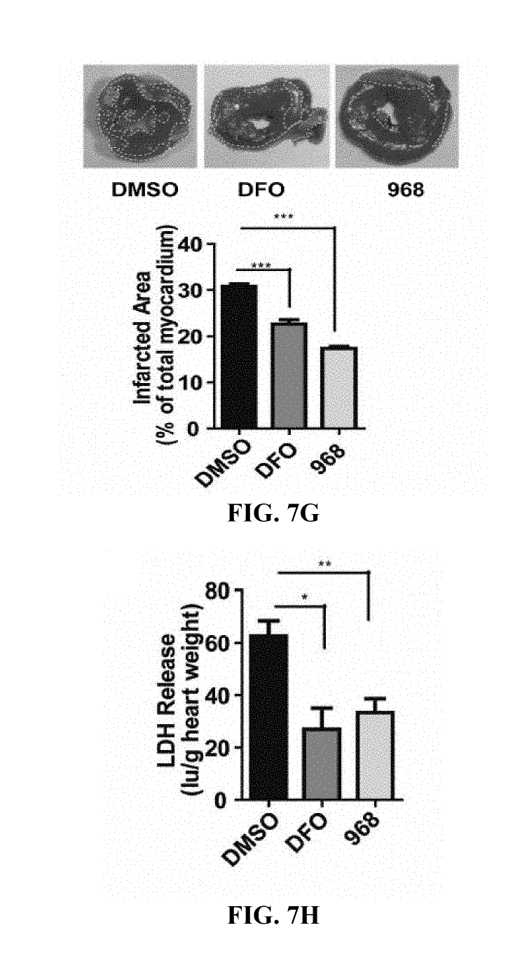

Requirement of cystine starvation, cellular glutathione depletion, and iron-carrier transferrin led us to test whether serum-dependent necrosis is the same as ferroptosis, a recently-discovered regulated necrosis process triggered by the synthetic chemical compound erastin and dependent on iron (Dixon et al., 2012; Dolma et al., 2003; Yagoda et al., 2007). Erastin triggers ferroptosis by inhibiting cystine uptake and thus depleting cellular glutathione (Dixon et al., 2012; Yang et al., 2014). The following experiments confirmed that serum-dependent necrosis is indeed ferroptosis, or at least these two modes of cell death share central mechanisms. First, ferrastatin-1 (Fer-1), a specific ferroptosis inhibitor (Dixon et al., 2012), can block serum-dependent cell death triggered by either full amino acid starvation or cystine starvation (FIG. 7A). Second, both transferrin and glutamine were required for erastin-induced ferroptosis (FIG. 7B). Further, as iron chelators (DFO and CPX), glutaminolysis inhibitors Compound 968 and AOA can also inhibit erastin-induced ferroptosis (FIGS. 7C-D), demonstrating that ferroptosis requires the cellular metabolic process glutaminolysis.

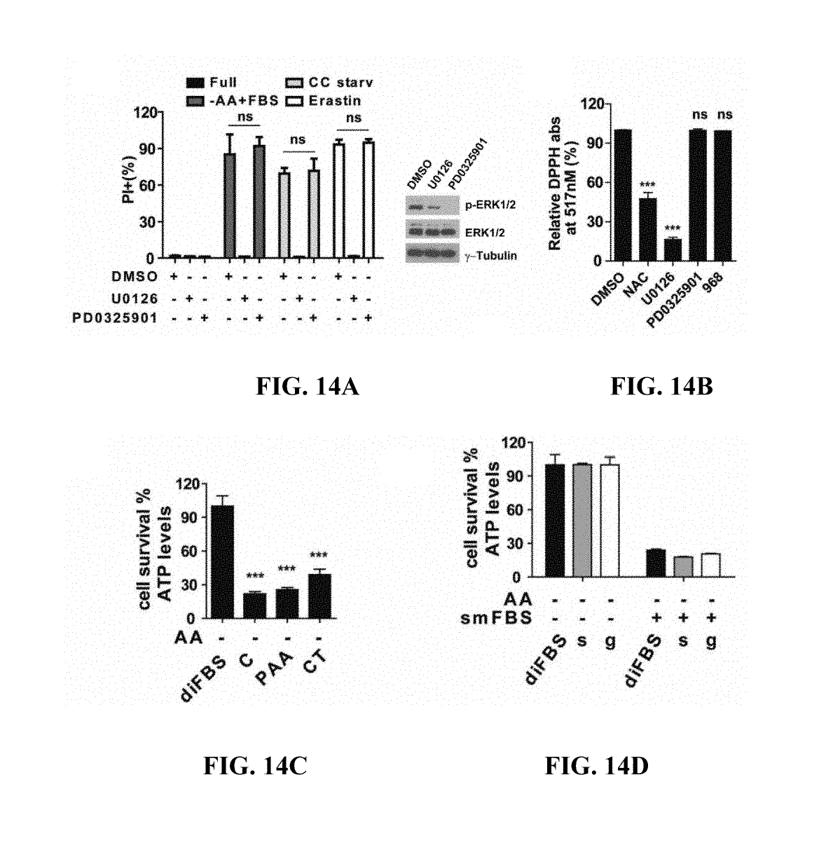

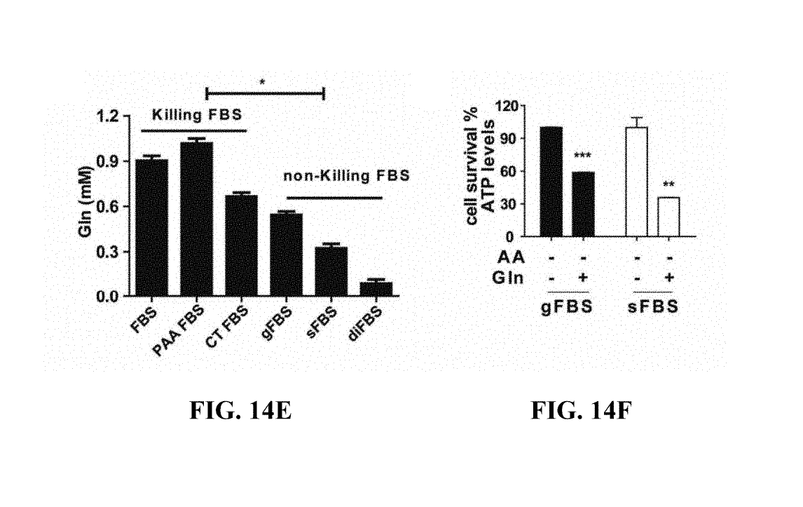

Previous studies suggest that ferroptosis depends on Ras-ERK signaling and can be completely blocked by MEK inhibition (Yagoda et al., 2007). We found that this conclusion is inaccurate, likely due to the use of MEK inhibitor U0126 in the previous studies. We compared U0126 with a more selective and potent MEK1/2 inhibitor, PD0325901, and found that U0126 but not PD0325901 is able to block cell death induced by either erastin or amino acid starvation in the presence of serum, albeit PD0325901 inhibited MEK activity more completely than U0126 in the experiments (FIG. 14A). Therefore, MEK activity is not essential for ferroptosis. The unintended effect of U0126 may be due to off-target inhibition of certain unknown enzymes required for ferroptosis, it might also be due to the anti-oxidative property of this compound as measured by its ability to reduce 2,2-Diphenyl-1-picrylhydrazyl (DPPH) (FIG. 14B). It should also be noted that PD0325901 and Compound 968 showed no anti-oxidant function (FIG. 14B).

Recent studies indicate that ferroptosis is associated with ischemia-reperfusion injury of organs such as liver and kidney, and is thus a potential therapeutic target (Friedmann Angeli et al., 2014; Linkermann et al., 2014). Since we identified glutaminolysis as a new essential factor for ferroptosis, we sought to test if the glutaminolysis inhibitor Compound 968 might also be able to reduce ischemia-reperfusion injuries in an ex vivo heart model. We subjected the hearts isolated from wild-type mice to ischemia-reperfusion stress (FIG. 7E). At the end of reperfusion, the hearts treated with either iron chelator DFO (a documented ferroptosis inhibitor (Dixon et al., 2012), serving as positive control) or GLS inhibitor Compound 968 showed significant improved function than hearts treated with vehicle (DMSO) as assessed by measuring the left ventricular developed pressure (LVDP) (FIG. 7F; vehicle 43.20%±1.66% of Baseline, DFO 64.75%±4.09% and Compound-968 75.25%±5.75%). This functional improvement was consistent with the reduction in myocardial infarcts size (FIG. 7G; vehicle treatment 30.82%±0.57% of myocardium, DFO treatment 22.60%±0.97%, and Compound-968 treatment 17.36%±0.47%). Similarly, DFO or compound-968 significantly inhibited the release of lactate dehydrogenase (LDH) during reperfusion, another indicator of myocardial injury (FIG. 7H). These data suggest that inhibition of ferroptosis via ablating glutaminolysis can protect heart tissue from ischemia-reperfusion injury.

Collectively, our study uncovered novel components and regulatory mechanisms for ferroptosis, a RIP3-independent programmed necrosis process. Extracellularly, serum components L-glutamine and transferrin have been identified as crucial regulators of ferroptosis. Intracellularly, the specific metabolic pathway glutaminolysis and components mediating transferrin import are required for ferroptosis. Several intriguing and seemingly counter-intuitive observations are associated with these new findings: under normal conditions, both transferrin and glutamine are required for cell survival and growth. However, upon amino acid starvation, these growth/survival factors function to unleash ferroptotic cell death that is more rapid and potent than that caused by amino acid starvation alone. Such novel function of transferrin and glutamine is reminiscent of the pro-death function of the life-essential protein cytochrome c in the intrinsic apoptotic pathway. Similarly, glutaminolysis is crucial for cellular biosynthesis and proliferation, but here it is actively involved in and essential for this intriguing metabolic cell death process.

This study raised many important mechanistic questions for further understanding of ferroptosis. How do transferrin, cellular glutaminolysis, glutathione synthesis, and cellular ROS generation process communicate with each other to achieve cell death, and what are the other molecular components or signaling pathways involved in this death process? Further, are the extracellular cues of ferroptosis regulated? There are a variety of potential mechanisms for such extracellular regulation. For example, we found that the killing activity of transferrin is dictated by its iron-loading status, thus mechanisms controlling iron-loading of transferrin may impact ferroptosis. Additionally, blood glutamine concentration can be variable. Indeed, when we used different FBS preparations, we found that death-inducing activity ranged from very potent, modest, to almost undetectable (FIGS. 14C, 14D). Strikingly, we found a positive correlation between the killing activity of individual FBS preparations and their L-Gln concentrations, and addition of L-Gln or the smFBS fraction prepared from death-competent FBS can restore the killing activity of those inactive FBS preparations (FIGS. 14D-14F).

Relevant to human disease, this study provided further evidence to support that ferroptosis is responsible, at least partially, for organ injury triggered by ischemia-reperfusion. This study also demonstrated that enzymes involved in glutaminolysis are potential therapeutic targets because of their crucial role in ferroptosis.

Accordingly, the present invention is directed to a method of treatment of an organ or a tissue injury caused by ischemia-reperfusion in a subject in need thereof, the method comprising administering to the subject a therapeutically effective amount of a ferroptosis inhibitor.

In another embodiment, the present invention is directed to a method of treatment of an organ or a tissue injury caused by ischemia-reperfusion in a subject in need thereof, the method comprising administering to the subject a therapeutically effective amount of a glutaminolysis inhibitor.

In another embodiment, the present invention is directed to a method of treatment of an organ or a tissue injury caused by ischemia-reperfusion in a subject in need thereof, the method comprising administering to the subject a therapeutically effective amount of a compound of formula (I):

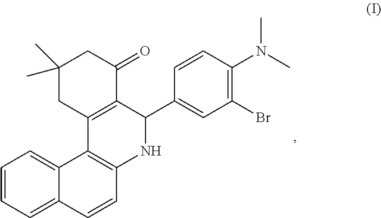 [SMILES: CN(C)c1ccc(C2Nc3ccc4ccccc4c3C3=C2C(=O)CC(C)(C)C3)cc1Br]

or a pharmaceutically acceptable salt thereof. The compound of formula (I) is compound 968 disclosed in Wang et al., (2010) as a glutaminolysis inhibitor. As explained above, we've discovered that the compound of formula (I) is also a ferroptosis inhibitor.

In one embodiment, the organ injury caused by ischemia-reperfusion is a brain injury, heart injury, renal injury, liver injury, or any combination thereof. In another embodiment, the organ injury caused by ischemia-reperfusion is a heart injury.

Ischemia-reperfusion causes injury in heart, liver, kidney, and brain, in part, by similar mechanisms. In all these organs, ischemia-reperfusion modulates changes in glutamine levels. Glutaminolysis is a major regulator of glutamine levels in these organs during injury. A glutaminolysis inhibitor would maintain higher levels of glutamine during ischemia-reperfusion, help promote energy metabolism and reduce oxidative stress, thus lessening injury. Therefore, administration of a glutaminolysis inhibitor would lessen injury to these organs. A ferroptosis inhibitor would also reduce oxidative stress, thus lessening injury. Therefore, administration of a ferroptosis inhibitor would also lessen injury to these organs. Administration of a glutaminolysis inhibitor and/or ferroptosis inhibitor may be performed prior to ischemia, during ischemia, and/or after ischemia.

In one embodiment, the present invention is directed to the use of the compound of formula (I) or to the use of a pharmaceutically acceptable salt thereof.

In one embodiment, the method of the present invention includes administering the ferroptosis inhibitor in the form of a composition comprising the ferroptosis inhibitor and a pharmaceutically acceptable buffer, diluent, carrier, adjuvant, or excipient.

In one embodiment, the method of the present invention includes administering the glutaminolysis inhibitor in the form of a composition comprising the glutaminolysis inhibitor and a pharmaceutically acceptable buffer, diluent, carrier, adjuvant, or excipient.

In one embodiment, the present invention is directed to a method for reducing the likelihood of an organ or a tissue injury caused by ischemia-reperfusion in a subject at risk or suspected of having the organ or the tissue injury, the method comprising administering to the subject a therapeutically effective amount of a compound of formula (I), or a pharmaceutically acceptable salt thereof.

In another embodiment, the present invention is direct to a use of a ferroptosis inhibitor in the treatment or prevention of an organ or a tissue injury caused by ischemia-reperfusion. The ferroptosis inhibitor may be a compound formula (I), or a pharmaceutically acceptable salt thereof.

In yet another embodiment, the present invention is direct to a use of a glutaminolysis inhibitor in the treatment or prevention of an organ or a tissue injury caused by ischemia-reperfusion. The glutaminolysis inhibitor may be the compound formula (I), or a pharmaceutically acceptable salt thereof.

In another embodiment, the present invention is directed to a method of treatment of an organ injury caused by ischemia-reperfusion in a subject in need thereof, the method comprising contacting the organ with a ferroptosis inhibitor or a glutaminolysis inhibitor. The compound of formula (I) may be the ferroptosis inhibitor or the glutaminolysis inhibitor. The organ injury caused by ischemia-reperfusion may be a brain injury, heart injury, renal injury, liver injury, or any combination thereof.

In another embodiment, the present invention is directed to a method of treatment of a tissue injury caused by ischemia-reperfusion in a subject in need thereof, the method comprising contacting the tissue with a ferroptosis inhibitor or a glutaminolysis inhibitor. The compound of formula (I) may be the ferroptosis inhibitor or the glutaminolysis inhibitor. The organ injury caused by ischemia-reperfusion may be a brain injury, heart injury, renal injury, liver injury, or any combination thereof.

In general the terms and phrases used herein have their art-recognized meaning, which can be found by reference to standard texts, journal references and contexts known to those skilled in the art. The following definitions are provided to clarify their specific use in the context of the invention.

As used herein, the phrase “pharmaceutically acceptable” indicates that the designated carrier, vehicle, diluent, excipient, salt or prodrug is generally chemically and/or physically compatible with the other ingredients comprising a formulation, and is physiologically compatible with the recipient thereof.

The terms “treating”, “treated”, and “treatment” as used herein include preventative (e.g., prophylactic), ameliorative, palliative and curative uses and/or results.

The phrases “therapeutic” and “therapeutically effective amount” as used herein denote an amount of a compound, composition or medicament that (a) treats or prevents a particular disease, condition or disorder; (b) attenuates, ameliorates or eliminates one or more symptoms of a particular disease, condition or disorder; (c) prevents or delays the onset of one or more symptoms of a particular disease, condition or disorder described herein. It should be understood that the terms “therapeutic” and “therapeutically effective” encompass any one of the aforementioned effects (a)-(c), either alone or in combination with any of the others (a)-(c).

The term “subject” as used herein denotes an animal, preferably a mammal. The term “mammal” is used in its dictionary sense. Humans are included in the group of mammals, and humans would be the preferred subjects.

A pharmaceutical composition of the invention, for example, includes forms suitable for oral administration as a tablet, capsule, pill, powder, sustained release formulations, solution, suspension, or for parenteral injection as a sterile solution, suspension or emulsion. Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

In one preferred embodiment, the compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations, such as tablets, capsules containing particulates, liquids, or powders; lozenges (including liquid-filled), chews; multi- and nano-particulates; gels, solid solution, liposome, films (including muco-adhesive), ovules, sprays and liquid formulations. Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet. The compounds of the invention may also be used in fast-dissolving, fast-disintegrating dosage forms such as those described in Expert Opinion in Therapeutic Patents, 11 (6), 981-986 by Liang and Chen (2001).

In another preferred embodiment, the compounds of the invention may be administered by parenteral injection. Exemplary parenteral administration forms include sterile solutions, suspensions or emulsions of the compounds of the invention in sterile aqueous media, for example, aqueous propylene glycol or dextrose. In another embodiment, the parenteral administration form is a solution. Such parenteral dosage forms can be suitably buffered, if desired.

Dosage regimens of the compounds and/or pharmaceutical composition of the invention may be adjusted to provide the optimum desired response. For example, a single bolus may be administered, several divided doses may be administered over time or the dose may be proportionally reduced or increased as indicated by the exigencies of the therapeutic situation. The appropriate dosing regimen, the amount of each dose administered and/or the intervals between doses will depend upon the compound of the invention being used, the type of pharmaceutical composition, the characteristics of the subject in need of treatment and the severity of the condition being treated.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient.

It should be noted that variation in the dosage will depend on the compound employed, the mode of administration, the treatment desired and the disorder (severity and type) to be treated or alleviated. The present invention also encompasses sustained release compositions and “flash” formulations, i.e. providing a medication to dissolve in the mouth.

It is to be further understood that for any particular subject, specific dosage regimens should be adjusted over time according to the individual need and the professional judgment of the person administering or supervising the administration of the compositions. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the chemotherapeutic agent are well-known in the relevant art and would be understood to be encompassed by the skilled artisan once provided the teachings disclosed herein.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, a pharmaceutical composition of the invention may comprise between 0.1% and 100% (w/w) active ingredient. In addition to the active ingredient, a pharmaceutical composition of the invention may further comprise one or more additional pharmaceutically active agents.

Further aspects of the present disclosure relate to a pharmaceutical composition comprising a therapeutically effective amount of ferroptosis inhibitor, or a pharmaceutically acceptable salt or pro-drug thereof, either alone or in combination with a second agent, and a pharmaceutically acceptable carrier, vehicle, diluent or excipient.

The pharmaceutical composition of the invention may comprise one or more other active agents, in which case ferroptosis inhibitor and the other agent(s) may be administered as part of the same or separate dosage forms, via the same or different routes of administration, and on the same or different administration schedules according to standard pharmaceutical practice.

Throughout this application, various references are referred to. The disclosures of these publications in their entireties are hereby incorporated by reference as if written herein.

The following specific non-limiting examples are illustrative of the invention.

### EXAMPLES

### Example 1

### Cell Culture

Unless specified otherwise, all mammalian cells are maintained in MEM medium with high-glucose, sodium pyruvate (1 mM), glutamine (2 mM), penicillin (U/ml), streptomycin (0.1mg/ml) and 10% (v/v) FBS at 37° C. and 5% CO₂.

### Example 2

### Induction and Measurement of Cell Death

To induce cell death, 80%-confluent cells were washed with PBS twice, and then cultured in amino acid-free medium, with specific factors added as indicated in individual experiments. Cell death was analyzed by propidium iodide (PI) staining coupled with microscopy or flow cytometry. Alternatively, cell viability was determined using the CellTiter-Glo luminescent Cell Viability Assay (Promega). In assays using WT MEFs, viability was calculated by normalizing ATP levels to cells treated with amino acid-starvation in the presence of 10% (v/v) diFBS, while in assays using bax/bak-DKO MEFs, ATP levels were normalized to cells treated with amino acid and FBS double starvation.

### Example 3

### Antibodies

Primary antibodies used were anti-bovine transferrin (BETHYL, Cat #A10-122A), anti-TfR (Life Science, Cat #136800), anti-Caspase3 (Cell Signaling, Cat #9665), anti-γ-Tubulin (Sigma, Cat #T6557), anti-GLS1 (Proteintech, Cat #12855-1), anti-GLS2 (Prosci, Cat #6217), anti-RW3 (Prosci, Cat #2283), anti-GLUD1 (Cell signaling, Cat #9828), sheep IgG (BETHYL, Cat #P130-100), anti-pERK1/2 (Cell Signaling, Cat #4370S), and anti-ERK1/2 (Cell Signaling, Cat #9107).

### Example 4

### Reagents

Compound 968 was purchased from Millipore (Cat #352010). MEK1/2 inhibitor (PD0325901) was purchased from Millipore (Cat #444966). U0126 was purchased from Cell Signaling Technology (Cat#9903) .zVAD was purchased from ENZO (Cat #ALX-260-020). Erastin was purchased from Millipore (Cat#329600). Ferrostatin-1 (Fer-1) was purchased from XcessBio (Cat#M60042). The source of commercial transferrins is as follows: bovine holo-transferrin (Sigma Cat #T1283), bovine apo-transferrin (Sigma Cat #T1428), and recombinant human holo-transferrin (Sigma Cat #T3705). All other chemicals were purchased from Sigma-Aldrich. Different Fetal Bovine Serum preparations used in this study are as follows: FBS (GEMINI, Cat #100-125; Lot #A51C05A), PAA FBS (PAA, Cat #A15-201; Lot #A20111-7008), CT FBS (Clontech, Cat #631106; Lot #A301097018), sFBS (sigma, Cat #F2442; Lot #12H045) and gFBS (Gibco, Cat #10437-028; Lot #1036512).

### Example 5

Purification and Identification of Transferrin from FBS

All purification steps were carried out at 4° C., and chromatography was performed with an Amersham FPLC system. For the purification, 20 ml FBS (664 mg protein) was applied to 50-70% (saturation) ammonium sulfate precipitation. The protein pellet (242 mg) that contained the activity was dissolved in 4 ml Buffer A (20 mM Hepes, PH 7.5 10 mM NaCl) and dialyzed overnight. The activity was applied to HiTrap SP Sepharose (GE Healthcare). The flow-through containing the activity was subjected to HiTrap Q Sepharose (GE Healthcare). After washing the column with Buffer A, the fractions was eluted by a gradient of 10-300 mM NaCl in Buffer A. Activity-containing fractions were further fractionated with HiTrap Heparin Sepharose (GE Healthcare) by using a gradient 10-300 mM NaCl in Buffer A. Fractions of 1 ml was collected. After dialysis, filtered with 0.2 mM filter, the fractions was assayed for activity. SDS-PAGE and Coomassie staining (Bio-Rad) was preformed, and a single band correlated with the killing activity was subject to protein identity determination by mass spectrometry analysis (MALDI-TOF-MS/MS). The activity was identified as bovine transferrin.

### Example 6

### Immuno-Depletion of Transferrin

To deplete transferrin from serum, amino acid-free DMEM medium containing 10% FBS was incubated with control IgG or anti-bovine transferrin antibody bound to Protein G Agarose (GE Healthcare) overnight at 4° C. Protein G Agarose was removed by centrifugation and the supernatant was assayed for killing activity.

### Example 7

Purification and Identification of L-Glutamine from FBS

FBS was filtered through Centrifugal Filter Units (MWCO 10 KDa) (Millipore) to obtain small molecule fraction (smFBS). One ml of smFBS was dried under vacuum and dissolved in 1 ml methanol and insoluble material was removed by centrifugation. The supernatant was dried and dissolved in 750-μl methanol first and then mixed with 614-μl acetonitrile (final ratio of methanol: acetonitrile is 55:45). After incubating the mix for 30 min at 4° C., precipitated material was removed by centrifugation, and the supernatant was dried and dissolved in 750-μl methanol. Aliquot of 150-μl was mixed with 1350-μl acetonitrile (final ratio of methanol: acetonitrile is 10:90) and incubated for 30 min at 4° C. The insoluble material was obtained by centrifugation and then dissolved in 75-μl ddH₂O. An aliquot of 50-μl as input was applied to a reversed phase XDB-C18 (4.6×250 mm) HPLC analytical column (Agilent). Separation was achieved by use of step elution consisting of A (ddH₂O) and B (methanol) as following: 0.00-11.00 min: 100% A, flow rate 1 ml/min; 11.00-21.00 min: 100% B, flow rate 1 ml/min. All fractions were dried and dissolved in 100-μl ddH₂O, and 25-μl of each fraction was subjected to activity assay. The fraction with the highest killing activity was subjected to mass spectrometry (PE SCIEX API-100 LC/MS system, mass range: 30.0 to 500.0 by amu).

### Example 8

### Quantitative RT-PCR

Total RNA was extracted using Aurum Total RNA mini kit (Bio-Rad) and reverse transcription was preformed from 400 ng of total RNA using iScript cDNA synthesis kit (Bio-Rad). Quantitative RT-PCR was performed with iQ SYBR Green Supermix (Bio-Rad) using a CFX connect Real-time System (Bio-Rad). The relative level of mRNA was calculated by comparative Ct method using Actin as control. The primers for SLC38A1 are SLC38A1-F: TGGTGACCATCATACTCTTG (SEQ ID No; 1) and SLC38a1-R: TCAGTGGCCTTCGTCGGTGC (SEQ ID NO; 2); the primers for Actin are: ACTIN-F: GGCACCACACCTTCTACAATG (SEQ ID NO; 3) and ACTIN-R: GGGGTGTTGAAGGTCTCAAAC (SEQ ID NO; 4); the primers for GCLC are GCLC-F: TGAGCATAGACACCATCATC (SEQ ID NO; 5) and GCLC-R: GGTAGTTCAGAATACTGCATC (SEQ ID NO; 6).

### Example 9

Lentiviral-Mediated shRNA Interference

MISSION lentiviral shRNA clones targeting mouse TfR, SLC38A1 GLUD1, GLS1, GLS2 and non-targeting control construct were purchased from Sigma-Aldrich. Lentivirus was packaged in 293T cells, and used to infect target cells which were then selected with puromycin for at least 3 days prior to use in experiments. The clones ID for the shRNA are TtR-sh1: TRCN0000375693, TtR-sh2 TRCN0000375695; SLC38A1 KD: TRCN0000069231; GLS1-sh1: TRCN0000253163, GLS-sh2: TRCN0000253167; GLS2-sh1: TRCN0000177027, GLS2-sh2: TRCN0000198217, GLUD1-sh1: TRCN0000041506, GLUD1-sh2: TRCN0000041507, GCLC KD: TRCN0000311454.

### Example 10

### Time-Lapse Microscopy

Live cell imaging of H2b-mcherry expressing MEFs was performed on glass-bottom 6-well plates (MatTek, Ashland, Mass.) using a Nikon Ti-E inverted microscope attached to a Coo1SNAP CCD camera (Photometrics). Fluorescence and differential interference contrast (DIC) images were acquired every 7 minutes, and images were analyzed using NIS elements software (Nikon) and ImageJ software (NIH). For confocal imaging, MEFs were grown on 35 mm glass bottom plates (MatTek, Ashland, Mass.) and DIC images were acquired every 5 minutes with the Ultraview Vox spinning disc confocal system (Perkin Elmer) equipped with a Yokogawa CSU-X1 spinning disc head, and EMCCD camera (Hamamatsu C9100-13), and coupled with a Nikon Ti-E microscope. Image analysis was performed with Volocity software (Perkin Elemer). All imaging was carried out in incubation chambers at 5% CO₂ and 37° C.

### Example 11

### Glutamine Concentration Measurement

The concentrations of glutamine in different FBS were measured by an YSI 7000 enzymatic analyzer according to the manual.

### Example 12

### GSH Mmeasurement

2×10⁵ MEFs were seeded in 6-well plates. One day later, cells were treated as indicated for 6 hours. Cells were harvested and cell numbers were determined. Total glutathione was measured as described previously (Rahman et al., 2006.)

### Example 13

### Measurement of Reactive Oxygen Species (ROS)

MEFs were treated as indicated, and then 10 μM 2′,7′-dichlorodihydrofluorescein diacetate (H2DCFDA, Life Technologies Cat#D-399) was added and incubated for 1 hour. Excess H2DCFDA was removed by washing the cells twice with PBS. Labeled cells were trypsinized and resuspended in PBS plus 5% FBS. Oxidation of H₂DCFDA to the highly fluorescent 2′,7′-dichlorofluorescein (DCF) is proportional to ROS generation and was analyzed using a flow cytometer (Fortessa, BD Biosciences). A minimum of 10,000 cells was analyzed per condition.

### Example 14

### 2,2-Diphenyl-1-Picrylhydrazyl Assay for Antioxidant Activity

The experiment was performed as described previously (Blois, 1958; Dixon et al., 2012). 2,2-Diphenyl-1-picrylhydrazyl (DPPH) (Sigma Cat#D9132) was dissolved in methanol to a final concentration of 50 μM. The tested compounds were added to 1 ml of DPPH solution with a final concentration of 50 μM. Samples were mixed well and incubated at room temperature for 1 hr. The absorbance at 517 nm (indicating the concentration of non-reduced DPPH) was measured using methanol as control. Results were normalized to DMSO (which has no antioxidant activity; set as 100%).

### Example 15

Ischemia-Reperfusion (I/R) Analysis using Isolated Hearts

Male C57BL/6J mice weighing 25-30 g at age 12-14 weeks were used in all experiments and maintained in a temperature-controlled room with alternating 12:12-h light-dark cycles. Experiments were performed using an isovolumic isolated heart preparation as published and modified for the use in mice hearts (Ananthakrishnan et al., 2009; Hwang et al., 2004). Hearts from 12-14 weeks aged wild-type mice were isolated, and retrograde perfused at 37° C. in a non-recirculating mode through the aorta at a rate of 2.5 ml/min. Hearts were perfused with modified Krebs-Henseleit (KH) buffer (118 mM NaCl, 4.7 mM KCl, 2.5 mM CaCl₂, 1.2 mM MgCl₂, 25 mM NaHCO₃, 5 mM glucose, 0.4 mM palmitate, 0.2 mM glutamine, 10 μg/ml human recombinant transferrin, 0.4 mM BSA, and 70 mU/1 insulin). Left ventricular developed pressure (LVDP) was measured using a latex balloon in the left ventricle. LVDP and coronary perfusion pressure were monitored continuously on a four-channel Gould recorder. Hearts were perfused either with KH buffer containing vehicle (DMSO) or the Compounds throughout the FR protocol. After an equilibration period of 30 min, global ischemia was performed for 30 min followed by 60 minutes of reperfusion. Cardiac injury due to I/R stress was assessed by measuring LDH release in the perfusates that were collected during 60 min of reperfusion. Infarct area was measured using 2,3,5-triphenyltetrazolium chloride (TTC) staining. After 60 min of reperfusion, the heart is perfused with Evans blue in-situ and then removed. Hearts were sliced into cross-sections at approximately 1-mm intervals. The sections were embedded in the TTC solution at 37° C. for 10 min, and area of infarct as a percent of the whole heart was quantified as described. Functional recovery of LVDP was expressed by comparing to the initial LVDP before ischemia. All animal experiments were approved by the Institutional Animal Care and Use Committees of New York University School of Medicine and conformed to the guidelines outlined in the National Institutes of Health Guide for Care and Use of Laboratory Animals (NIH Pub. No. 85-23, 1996).

### Example 16

### Statistical Analyses

All statistical analyses were performed using Prism 5.0c GraphPad Software. P values were calculated with unpaired Student's t test.

### REFERENCES

Ananthakrishnan, R., Kaneko, M., Hwang, Y. C., Quadri, N., Gomez, T., Li, Q., Caspersen, C., and Ramasamy, R. (2009). Aldose reductase mediates myocardial ischemia-reperfusion injury in part by opening mitochondrial permeability transition pore. American journal of physiology. Heart and circulatory physiology 296, H333-341.

Andrews, N. C., and Schmidt, P. J. (2007). Iron homeostasis. Annual review of physiology 69, 69-85.

Bergsbaken, T., Fink, S. L., and Cookson, B. T. (2009). Pyroptosis: host cell death and inflammation. Nat Rev Microbiol 7, 99-109.

Blois, M. S. (1958). Antioxidant Determinations by the Use of a Stable Free Radical. Nature 181, 1199-1200.

Blum, E. S., Abraham, M. C., Yoshimura, S., Lu, Y., and Shaham, S. (2012). Control of Nonapoptotic Developmental Cell Death in Caenorhabditis elegans by a Polyglutamine-Repeat Protein. Science 335, 970-973.

Budihardjo, I., Oliver, H., Lutter, M., Luo, X., and Wang, X. (1999). Biochemical pathways of caspase activation during apoptosis. Annual review of cell and developmental biology 15, 269-290.

Cho, Y. S., Challa, S., Moquin, D., Genga, R., Ray, T. D., Guildford, M., and Chan, F. K. (2009). Phosphorylation-driven assembly of the RIP1-RIP3 complex regulates programmed necrosis and virus-induced inflammation. Cell 137, 1112-1123.

Curthoys, N. P., and Watford, M. (1995). Regulation of glutaminase activity and glutamine metabolism. Annual review of nutrition 15, 133-159.

Danial, N. N., and Korsmeyer, S. J. (2004). Cell death: Critical control points. Cell 116, 205-219.

DeBerardinis, R. J., Lum, J. J., Hatzivassiliou, G., and Thompson, C. B. (2008). The biology of cancer: metabolic reprogramming fuels cell growth and proliferation. Cell metabolism 7, 11-20.

Degterev, A., Hitomi, J., Germscheid, M., Ch'en, I. L., Korkina, O., Teng, X., Abbott, D., Cuny, G. D., Yuan, C., Wagner, G., et al. (2008). Identification of RIP1 kinase as a specific cellular target of necrostatins. Nature chemical biology 4, 313-321.

Dixon, S. J., Lemberg, K. M., Lamprecht, M. R., Skouta, R., Zaitsev, E. M., Gleason, C. E., Patel, D. N., Bauer, A. J., Cantley, A. M., Yang, W. S., et al. (2012). Ferroptosis: an iron-dependent form of nonapoptotic cell death. Cell 149, 1060-1072.

Dolma, S., Lessnick, S. L., Hahn, W. C., and Stockwell, B. R. (2003). Identification of genotype-selective antitumor agents using synthetic lethal chemical screening in engineered human tumor cells. Cancer cell 3, 285-296.

Esslinger, C. S., Cybulski, :K. A., and Rhoderick, J. F. (2005). Ngamma-aryl glutamine analogues as probes of the ASCT2 neutral amino acid transporter binding site. Bioorganic & medicinal chemistry 13, 1111-1118.

Friedmann Angeli, J. P., Schneider, M., Proneth, B., Tyurina, Y. Y.,Tyurin, V. A., Hammond, V. J., Herbach, N., Aichler, M., Walch, A., Eggenhofer, E., et al. (2014). Inactivation of the ferroptosis regulator Gpx4 triggers acute renal failure in mice. Nature cell biology 16, 1180-1191.

Fuchs, Y., and Steller, H. (2011). Programmed Cell Death in Animal Development and Disease. Cell 147, 742-758.

Green, D. R., and Kroemer, G. (2004). The pathophysiology of mitochondrial cell death. Science 305, 626-629.

He, S., Wang, L., Miao, L., Wang, T., Du, F., Zhao, L., and Wang, X. (2009). Receptor interacting protein kinase-3 determines cellular necrotic response to TNF-alpha. Cell 137, 1100-1111.

Hensley, C. T., Wasti, A. T., and DeBerardinis, R. J. (2013). Glutamine and cancer: cell biology, physiology, and clinical opportunities. The Journal of clinical investigation 123, 3678-3684.

Hwang, Y. C., Kaneko, M., Bakr, S., Liao, H., Lu, Y., Lewis, E. R., Yan, S., Ii, S., Itakura, M., Rui, L., et al. (2004). Central role for aldose reductase pathway in myocardial ischemic injury. FASEB journal: official publication of the Federation of American Societies for Experimental Biology 18, 1192-1199.

Kaiser, W. J., Upton, J. W., Long, A. B., Livingston-Rosanoff, D., Daley-Bauer, L. P., Hakem, R., Caspary, T., and Mocarski, E. S. (2011). RIP3 mediates the embryonic lethality of caspase-8-deficient mice. Nature 471, 368-372.

Linkermann, A., Skouta, R., Himmerkus, N., Mulay, S. R., Dewitz, C., De Zen, F., Prokai, A., Zuchtriegel, G., Krombach, F., Welz, P. S., et al. (2014). Synchronized renal tubular cell death involves ferroptosis. Proceedings of the National Academy of Sciences of the United States of America 111, 16836-16841.

McGivan J. D., and Bungard, C. I. (2007). The transport of glutamine into mammalian cells. Frontiers in bioscience: a journal and virtual library 12, 874-882.

Moriwaki, K., and Chan, F. K. (2013). RIP3: a molecular switch for necrosis and inflammation. Genes & development 27, 1640-1649.

Murphy, J. M., Czabotar, P. E., Hildebrand, J. M., Lucet, I. S., Zhang, J. G., Alvarez-Diaz, S., Lewis, R., Laiaoui, N., Metcalf, D., Webb, A. I., et al. (2013). The pseudokinase MLKL mediates necroptosis via a molecular switch mechanism. Immunity 39, 443-453.

Newton, K., Dugger, D. L., Wickliffe, K. E., Kapoor, N., de Almagro, M. C., Vucic, D., Komuves, L., Ferrando, R. E., French, D. M., Webster, J., et al. (2014). Activity of protein kinase RIPK3 determines whether cells die by necroptosis or apoptosis. Science 343, 1357-1360.

Oberst, A., Dillon, C. P., Weinlich, R., McCormick, L. L., Fitzgerald, P., Pop, C., Hakem, R., Salvesen, G. S., and Green, D. R. (2011). Catalytic activity of the caspase-8-FLIP(L) complex inhibits RIPK3-dependent necrosis. Nature 471, 363-367.

Rahman, I., Kode, A., and Biswas, S. K. (2006). Assay for quantitative determination of glutathione and glutathione disulfide levels using enzymatic recycling method. Nature protocols 1, 3159-3165.

Robinson, M. M., McBryant, S. J., Tsukamoto, T., Rojas, C., Ferraris, D. V., Hamilton, S. K., Hansen, J. C., and Curthoys, N. P. (2007). Novel mechanism of inhibition of rat kidney-type glutaminase by bis-2-(5-phenylacetamido-1,2,4-thiadiazol-2-yl)ethyl sulfide (BPTES). The Biochemical journal 406, 407-414.

Sun, L., Wang, H., Wang, Z., He, S., Chen, S., Liao, D., Wang, L., Yan, Liu, W., Lei, X., et al. (2012). Mixed lineage kinase domain-like protein mediates necrosis signaling downstream of RIP3 kinase. Cell 148, 213-227.

Thompson, C. B. (1995). Apoptosis in the Pathogenesis and Treatment of Disease. Science 267, 1456-1462.

Valaitis, A. P., and Theil, E. C. (1984). Developmental-Changes in Plasma Transferrin Concentrations Related to Red-Cell Ferritin. Journal of Biological Chemistry 259, 779-784.

Vanden Berghe, T., Linkermann, A., Jouan-Lanhouet, S., Walczak, H., and Vandenabeele, P. (2014). Regulated necrosis: the expanding network of non-apoptotic cell death pathways. Nature reviews. Molecular cell biology 15, 135-147.

Vandenabeele, P., Declercq, W., Van Herreweghe, F., and Vanden Berghe, T. (2010). The Role of the Kinases RIP1 and RIP3 in TNF-Induced Necrosis. Sci Signal 3.

Wang, J. B., Erickson, J. W., Fuji, R., Ramachandran, S., Gao, P., Dinavahi, R., Wilson, K. F., Ambrosio, A. L., Dias, S. M., Dang, C. V., et al. (2010). Targeting mitochondrial glutaminase activity inhibits oncogenic transformation. Cancer cell 18, 207-219.

Wei, M.C., Zong, W. X., Cheng, E. H., Lindsten, T., Panoutsakopoulou, V., Ross, A. J., Roth, K. A., MacGregor, G. R., Thompson, C. B., and Korsmeyer, S. J. (2001). Proapoptotic BAX and BAK: a requisite gateway to mitochondrial dysfunction and death. Science 292, 727-730.

Wise, D. R., DeBerardinis, R. J., Mancuso, A., Sayed, N., Zhang, X. Y., Pfeiffer, H. K., Nissim, I., Daikhin, E., Yudkoff, M., McMahon, S. B., et al. (2008). Myc regulates a transcriptional program that stimulates mitochondrial glutaminolysis and leads to glutamine addiction. Proceedings of the National Academy of Sciences of the United States of America 105, 18782-18787.

Yagoda, N., von Rechenberg, M., Zaganjor, E., Bauer, A. J., Yang, W. S., Fridman, D. J., Wolpaw, A. J., Smukste, I., Peltier, J. M., Boniface, J. J., et al. (2007). RAS-RAF-MFK-dependent oxidative cell death involving voltage-dependent anion channels. Nature 447. 864-868.

Yang, W. S., SriRamaratnam, R., Welsch, M. E., Shimada, K., Skouta, R., Viswanathan, V. S., Cheah, J. H., Clemons, P. A., Shamji, A. F., Clish, C. B., et al. (2014). Regulation of ferroptotic cancer cell death by GPX4. Cell 156, 317-331.

Yuan, J., and Kroemer, G. (2010). Alternative cell death mechanisms in development and beyond. Genes & development 24, 2592-2602.

Zhang, D. W., Shao, J., Lin, J Zhang, N., Lu, B. J., Lin, S. C., Dong, M. Q., and Han, J. (2009). RIPS, an energy metabolism regulator that switches TNF-induced cell death from apoptosis to necrosis. Science 325, 332-336.

What is claimed is:

1. A method of treatment of an organ or a tissue injury caused by ischemia-reperfusion in a subject in need thereof, the method comprising administering to the subject a therapeutically effective amount of a ferroptosis inhibitor.

2. The method of claim 1, wherein the organ injury caused by ischemia-reperfusion is a brain injury.

3. The method of claim 1, wherein the organ injury caused by ischemia-reperfusion is a heart injury.

4. The method of claim 1, wherein the organ injury caused by ischemia-reperfusion is a renal injury.

5. The method of claim 1, wherein the organ injury caused by ischemia-reperfusion is a liver injury.

6. The method of claim 1, wherein the ferroptosis inhibitor is a compound of formula (I): 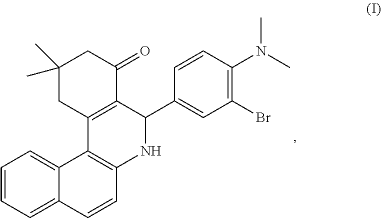 [SMILES: CN(C)c1ccc(C2Nc3ccc4ccccc4c3C3=C2C(=O)CC(C)(C)C3)cc1Br]
or a pharmaceutically acceptable salt thereof.

7. The method of claim 1, comprising administering the ferroptosis inhibitor in the form of a composition comprising the ferroptosis inhibitor and a pharmaceutically acceptable buffer, diluent, carrier, adjuvant, or excipient.

8. The method of claim 1, wherein the subject is a mammal.

9. The method of claim 1, wherein the subject is a human.

10. A method of treatment of an organ or a tissue injury caused by ischemia-reperfusion in a subject in need thereof, the method comprising administering to the subject a therapeutically effective amount of a compound of formula (I): 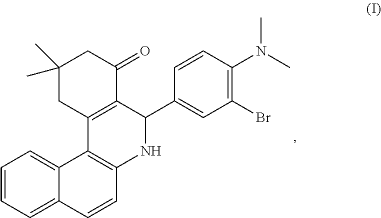 [SMILES: CN(C)c1ccc(C2Nc3ccc4ccccc4c3C3=C2C(=O)CC(C)(C)C3)cc1Br]
or a pharmaceutically acceptable salt thereof.

11. The method of claim 10, wherein the organ injury caused by ischemia-reperfusion is a brain injury.

12. The method of claim 10, wherein the organ injury caused by ischemia-reperfusion is a heart injury.

13. The method of claim 10, wherein the organ injury caused by ischemia-reperfusion is a renal injury.

14. The method of claim 10, wherein the organ injury caused by ischemia-reperfusion is a liver injury.

15. The method of claim 10, comprising administering the compound of formula (I) or a pharmaceutically acceptable salt thereof in the form of a composition comprising the compound of formula (I) or a pharmaceutically acceptable salt thereof and a pharmaceutically acceptable buffer, diluent, carrier, adjuvant, or excipient.

16. The method of claim 10, wherein the subject is a mammal.

17. The method of claim 10, wherein the subject is a human.

18. A method for reducing the likelihood of an organ or a tissue injury caused by ischemia-reperfusion in a subject at risk or suspected of having the organ or the tissue injury, the method comprising administering to the subject a therapeutically effective amount of a compound of formula (I): 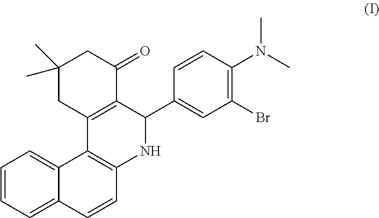 [SMILES: CN(C)c1ccc(C2Nc3ccc4ccccc4c3C3=C2C(=O)CC(C)(C)C3)cc1Br]
or a pharmaceutically acceptable salt thereof.

19. A method of treatment of an organ or a tissue injury caused by ischemia-reperfusion in a subject in need thereof, the method comprising administering to the subject a therapeutically effective amount of a glutaminolysis inhibitor.

20. The method of claim 21, wherein the organ injury caused by ischemia-reperfusion is a brain injury.

21. The method of claim 21, wherein the organ injury caused by ischemia-reperfusion is a heart injury.

22. The method of claim 21, wherein the organ injury caused by ischemia-reperfusion is a renal injury.

23. The method of claim 21, wherein the organ injury caused by ischemia-reperfusion is a liver injury.

24. The method of claim 21, wherein the glutaminolysis inhibitor is a compound of formula (I):
(I) 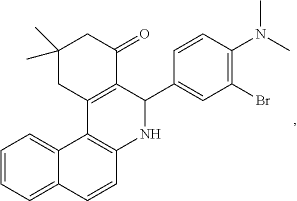 [SMILES: CN(C)c1ccc(C2Nc3ccc4ccccc4c3C3=C2C(=O)CC(C)(C)C3)cc1Br]
or a pharmaceutically acceptable salt thereof.

25. The method of claim 21, comprising administering the glutaminolysis inhibitor in the form of a composition comprising the glutaminolysis inhibitor and a pharmaceutically acceptable buffer, diluent, carrier, adjuvant, or excipient.

26. The method of claim 21, wherein the subject is a mammal.

27. The method of claim 21, wherein the subject is a human.

28. A method of treatment of an organ injury caused by ischemia-reperfusion in a subject in need thereof, the method comprising contacting the organ with a ferroptosis inhibitor.

29. A method of treatment of an organ injury caused by ischemia-reperfusion in a subject in need thereof, the method comprising contacting the organ with a glutaminolysis inhibitor.

30. A method of treatment of a tissue injury caused by ischemia-reperfusion in a subject in need thereof, the method comprising contacting the tissue with a ferroptosis inhibitor.

31. A method of treatment of a tissue injury caused by ischemia-reperfusion in a subject in need thereof, the method comprising contacting the tissue with a glutaminolysis inhibitor.


SEQUENCE LISTING (6 sequences)

SEQ ID NO: 1 - DNA, 20 bp, Artificial Sequence
  Synthetic Sequence
tggtgaccatcatactcttg

SEQ ID NO: 2 - DNA, 20 bp, Artificial Sequence
  Synthetic Sequence
tcagtggccttcgtcggtgc

SEQ ID NO: 3 - DNA, 21 bp, Artificial Sequence
  Synthetic Sequence
ggcaccacaccttctacaatg

SEQ ID NO: 4 - DNA, 21 bp, Artificial Sequence
  Synthetic Sequence
ggggtgttgaaggtctcaaac

SEQ ID NO: 5 - DNA, 20 bp, Artificial Sequence
  Synthetic Sequence
tgagcatagacaccatcatc

SEQ ID NO: 6 - DNA, 21 bp, Artificial Sequence
  Synthetic Sequence
ggtagttcagaatactgcatc